(19) United States
(12) Patent Application Publication
(10) Pub. No.: US 2025/0031995 A1
(43) Pub. Date: Jan. 30, 2025

(54) DYNAMIC BALANCE ESTIMATION DEVICE, DYNAMIC BALANCE ESTIMATION SYSTEM, DYNAMIC BALANCE ESTIMATION METHOD, AND RECORDING MEDIUM

(71) Applicant: NEC Corporation, Minato-ku, Tokyo (JP)

(72) Inventors: Chenhui HUANG, Tokyo (JP); Fumiyuki NIHEY, Tokyo (JP); Zhenwei WANG, Tokyo (JP); Hiroshi KAJITANI, Tokyo (JP); Yoshitaka NOZAKI, Tokyo (JP); Kenichiro FUKUSHI, Tokyo (JP)

(73) Assignee: NEC Corporation, Minato-ku, Tokyo (JP)

(21) Appl. No.: 18/716,561

(22) PCT Filed: Dec. 27, 2021
(86) PCT No.: PCT/JP2021/048550

§ 371 Date: Jun. 5, 2024

(51) Int. Cl. A61B 5/11

(52) U.S. Cl. CPC A61B 5/11

(57) ABSTRACT

Provided is a dynamic balance estimation device that includes a data acquisition unit that acquires feature amount data including a feature amount to be used for estimating dynamic balance of a user, the feature amount data being extracted from sensor data regarding motion of a foot of the user, a storage unit that stores an estimation model that outputs a dynamic balance index according to an input of the feature amount data, an estimation unit that inputs the acquired feature amount data to the estimation model to estimate the dynamic balance of the user in accordance with the dynamic balance index output from the estimation model, and an output unit that outputs information on the estimated dynamic balance of the user.

### TECHNICAL FIELD

The present disclosure relates to a dynamic balance estimation device, and the like, that estimate dynamic balance using sensor data regarding motion of a foot.

### BACKGROUND ART

With increasing interest in healthcare, services for providing information according to features (also referred to as gait) included in a gait pattern have attracted attention. For example, a technique for analyzing gait based on sensor data measured by a sensor mounted on footwear such as shoes has been developed. In time-series data of the sensor data, features of a gait event (also referred to as a gait event) related to a physical condition appear.

PTL 1 discloses a device that detects an abnormality of a foot based on features of gait of a walker. The device of PTL 1 extracts a characteristic gait feature amount in gait of the walker wearing footwear using data acquired from a sensor installed on the footwear. The device of PTL 1 detects an abnormality of the walker walking while wearing footwear based on the extracted gait feature amount. For example, the device of PTL 1 extracts a characteristic part regarding hallux valgus from gait waveform data for one gait cycle. The device of PTL 1 estimates a progress state of the hallux valgus using the gait feature amount of the extracted characteristic part.

PTL 2 discloses an evaluation method for collecting data of a plurality of users in a unified manner from information obtained from daily gait motion of the users. In the evaluation method of PTL 2, data of plantar pressures for a predetermined period during gait and during standing still is acquired using sensors provided in insoles of shoes used by the plurality of users. In the evaluation method of PTL 2, the acquired data is analyzed, and a plantar pressure parameter, a foot pressure center parameter, and a period parameter during gait, and a plantar pressure parameter and a foot pressure center parameter during standing still are acquired and accumulated for each user. PTL 2 discloses that user data having items (attributes) such as gender, date of birth, height, and weight is held in a database.

In order to maintain dynamic stability of gait, a dynamic balance ability corresponding to an ability to balance against sudden disturbances is important. The dynamic balance ability is an important indicator for assessing frailty and fall risk. The dynamic balance ability can be evaluated by a result of a functional reach test (FRT). The result of the FRT is evaluated by a distance (also referred to as an FR distance) from fingertips in a state where both hands are raised 90 degrees with respect to a horizontal plane to fingertips in a state where the upper limb is moved (reached) forward as much as possible.

NPL 1 reports a verification result in which muscle activity amounts of the multifidus muscle, the long head of the biceps femoris muscle, the soleus muscle, and the medial head of the flexor hallucis brevis muscle increase during functional reach motion. NPL 2 indicates a result in which a correlation is recognized between the hip joint abduction muscle and the FR distance. NPL 3 reports that, as a result of verification of a multidirectional FRT, there is a high correlation between muscle strength of the iliopsoas and the FR distance. NPL 4 discloses a consideration that stability is acquired by changing a foot angle as compensatory action for decrease in the balance ability and the muscle function associated with aging.

### CITATION LIST

### Patent Literature

PTL 1: WO 2021/140658 A1
PTL 2: JP 2021-166898 A

### Non Patent Literature

NPL 1: Kentaro Sasaki et al., “Electromyographic Analysis of ‘Functional Reach’”, Physical Therapy Science, Vol. 24 (6), pp. 813-816, 2009.
NPL 2: Takayuki Hirano, et al., “Relationship between Muscle Strength and Skeletal Muscle Mass and Physical Function among Community-Dwelling Elderly”, Nagoya School of Medicine, Health Science, and Sports Science, Vol. 4 (2), pp. 23-33, 2016.
NPL 3: Akira Ono, et al., “The relationship between the lower limbs and the functional reach in older adults”, Physical Exercise Measurement Evaluation Research, Vol. 1, pp. 119-126, 2001.
NPL 4: Yasuhiro Shirao, “Relationship between Foot Angle during Gait and Lower Limb Torsion Angle”, Physical Therapy Supplement, Vol. 41 Suppl. No. 2 (Abstract of the 49 Japan Physical Therapy Academic Meeting), pp. 0433, 2014.

### SUMMARY OF INVENTION

### Technical Problem

In the method of PTL 1, a progress state of the hallux valgus is estimated using the gait feature amount of the characteristic part extracted from the data acquired from the sensor installed in the footwear. PTL 1 does not disclose estimating dynamic balance using the gait feature amount of the characteristic part extracted from the data acquired from the sensor installed in the footwear.

In the method of PTL 2, parameters such as a plantar pressure parameter, a foot pressure center parameter, and a period parameter during gait are acquired and accumulated using data of a plantar pressure for the predetermined period measured by the sensor provided in the insole. In the method of PTL 2, a physical condition of the user is estimated by analyzing the accumulated parameters. PTL 2 discloses retaining attributes such as gender, date of birth, height, and weight, but does not disclose specific applications.

If the result of the FRT can be evaluated as in NPL 1 to NPL 3, dynamic balance can be evaluated. However, NPL 1 to NPL 3 does not disclose a method for evaluating the dynamic balance such as the FRT in daily life.

An object of the present disclosure is to provide a dynamic balance estimation device, and the like, that can appropriately estimate dynamic balance in daily life.

### Solution to Problem

A dynamic balance estimation device according to one aspect of the present disclosure includes a data acquisition unit that acquires feature amount data including a feature amount to be used for estimating dynamic balance of a user, the feature amount data being extracted from sensor data regarding motion of a foot of the user, a storage unit that stores an estimation model that outputs a dynamic balance index according to an input of the feature amount data, an estimation unit that inputs the acquired feature amount data to the estimation model to estimate the dynamic balance of the user in accordance with the dynamic balance index output from the estimation model, and an output unit that outputs information on the estimated dynamic balance of the user.

A dynamic balance estimation method according to one aspect of the present disclosure includes acquiring feature amount data including a feature amount to be used for estimating dynamic balance of a user, the feature amount data being extracted from sensor data regarding motion of a foot of the user, inputting the acquired feature amount data to an estimation model that outputs a dynamic balance index according to an input of the feature amount data, estimating the dynamic balance of the user in accordance with the dynamic balance index output from the estimation model, and outputting information on the estimated dynamic balance of the user.

A program according to one aspect of the present disclosure causes a computer to execute processing of acquiring feature amount data including a feature amount to be used for estimating dynamic balance of a user, the feature amount data being extracted from sensor data regarding motion of a foot of the user, processing of inputting the acquired feature amount data to an estimation model that outputs a dynamic balance index according to an input of the feature amount data, processing of estimating the dynamic balance of the user in accordance with the dynamic balance index output from the estimation model, and processing of outputting information on the estimated dynamic balance of the user.

### Advantageous Effects of Invention

According to the present disclosure, it is possible to provide a dynamic balance estimation device, and the like, that can appropriately estimate dynamic balance in daily life.

### BRIEF DESCRIPTION OF DRAWINGS

FIG. 1 is a block diagram illustrating an example of a configuration of a dynamic balance estimation system according to a first example embodiment.

FIG. 2 is a block diagram illustrating an example of a configuration of a gait measurement device included in the dynamic balance estimation system according to the first example embodiment.

FIG. 3 is a conceptual diagram illustrating an arrangement example of the gait measurement device according to the first example embodiment.

FIG. 4 is a conceptual diagram for explaining an example of a relationship between a local coordinate system and a world coordinate system set in the gait measurement device according to the first example embodiment.

FIG. 5 is a conceptual diagram for explaining a human body surface to be used in explanation regarding the gait measurement device according to the first example embodiment.

FIG. 6 is a conceptual diagram for explaining a gait cycle to be used in the explanation regarding the gait measurement device according to the first example embodiment.

FIG. 7 is a conceptual diagram for explaining gait parameters to be used in the explanation regarding the gait measurement device according to the first example embodiment.

FIG. 8 is a graph for explaining an example of time-series data of sensor data measured by the gait measurement device according to the first example embodiment.

FIG. 9 is a view for explaining an example of normalization of gait waveform data extracted from the time-series data of the sensor data measured by the gait measurement device according to the first example embodiment.

FIG. 10 is a conceptual diagram for explaining an example of a gait phase cluster from which a feature amount data generation unit of the gait measurement device according to the first example embodiment extracts a feature amount.

FIG. 11 is a block diagram illustrating an example of a configuration of a dynamic balance estimation device included in the dynamic balance estimation system according to the first example embodiment.

FIG. 12 is a conceptual diagram for explaining a functional reach distance to be estimated by the dynamic balance estimation system according to the first example embodiment.

FIG. 13 is a conceptual diagram for explaining the functional reach distance to be estimated by the dynamic balance estimation system according to the first example embodiment.

FIG. 14 is a table regarding specific examples of the feature amounts to be extracted by the gait measurement device included in the dynamic balance estimation system according to the first example embodiment to estimate a functional reach distance.

FIG. 15 is a graph indicating a correlation between a feature amount F1 extracted by the gait measurement device included in the dynamic balance estimation system according to the first example embodiment and a measured functional reach distance.

FIG. 16 is a graph indicating a correlation between a feature amount F2 extracted by the gait measurement device included in the dynamic balance estimation system according to the first example embodiment and a measured functional reach distance.

FIG. 17 is a graph indicating a correlation between a feature amount F3 extracted by the gait measurement device included in the dynamic balance estimation system according to the first example embodiment and a measured functional reach distance.

FIG. 18 is a graph indicating a correlation between a feature amount F4 extracted by the gait measurement device included in the dynamic balance estimation system according to the first example embodiment and a measured functional reach distance.

FIG. 19 is a graph indicating a correlation between a feature amount F5 extracted by the gait measurement device included in the dynamic balance estimation system according to the first example embodiment and a measured functional reach distance.

FIG. 20 is a block diagram illustrating an estimation example of the functional reach distance (dynamic balance index) by a dynamic balance estimation device included in the dynamic balance estimation system according to the first example embodiment.

FIG. 21 is a graph indicating a correlation between an estimated value of the functional reach distance estimated using an estimation model generated by machine learning using gender, age, height, weight, and gait speed as explanatory variables and a measured value of the functional reach distance.

FIG. 22 is a graph indicating a correlation between an estimated value of the functional reach distance estimated by the dynamic balance estimation device included in the dynamic balance estimation system according to the first example embodiment and a measured value of the functional reach distance.

FIG. 23 is a flowchart for explaining an example of operation of the gait measurement device included in the dynamic balance estimation system according to the first example embodiment.

FIG. 24 is a flowchart for explaining an example of operation of the dynamic balance estimation device included in the dynamic balance estimation system according to the first example embodiment.

FIG. 25 is a conceptual diagram for explaining an application example of the dynamic balance estimation system according to the first example embodiment.

FIG. 26 is a block diagram illustrating an example of a configuration of a machine learning system according to a second example embodiment.

FIG. 27 is a block diagram illustrating an example of a configuration of a machine learning device included in the machine learning system according to the second example embodiment.

FIG. 28 is a conceptual diagram for explaining an example of machine learning by the machine learning device included in the machine learning system according to the second example embodiment.

FIG. 29 is a block diagram illustrating an example of a configuration of a dynamic balance estimation device according to a third example embodiment.

FIG. 30 is a block diagram illustrating an example of a hardware configuration that executes control and processing according to each example embodiment.

### EXAMPLE EMBODIMENT

Hereinafter, example embodiments of the present invention will be described with reference to the drawings. However, the example embodiments described below have technically preferable limitations for carrying out the present invention, but the scope of the invention is not limited to the following. In all the drawings to be used in the following description of the example embodiments, the same reference numerals are given to the same parts unless there is a particular reason. Further, in the following example embodiments, repeated description of similar configurations and operation may be omitted.

### First Example Embodiment

First, a dynamic balance estimation system according to a first example embodiment will be described with reference to the drawings. The dynamic balance estimation system according to the present example embodiment measures sensor data regarding motion of a foot according to gait of a user. The dynamic balance estimation system of the present example embodiment estimates dynamic balance of the user using the measured sensor data.

In the present example embodiment, an example will be described where a result of a functional reach test (FRT) is estimated as the dynamic balance. In the present example embodiment, the result of the FRT is evaluated by a distance (also referred to as a functional reach distance) from fingertips in a state where both hands are raised by 90 degrees with respect to a horizontal plane and upright to fingertips in a state where the upper limb is moved (reached) forward as much as possible. The functional reach distance (hereinafter, referred to as an FR distance) is a value of the result of the FRT. The greater the FR distance, the higher the result of the FRT. The method of the present example embodiment can be applied to other than the FRT performed with both hands. For example, the method of the present example embodiment can be applied to the FRT performed with one hand or other variations of the FRT.

### (Configuration)

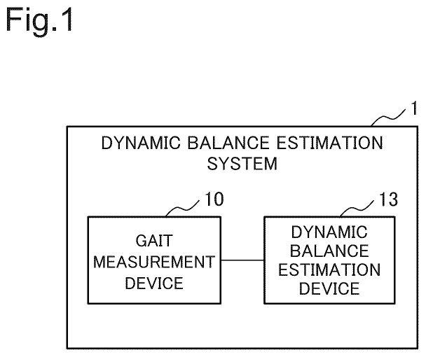

FIG. 1 is a block diagram illustrating an example of a configuration of a dynamic balance estimation system 1 according to the present example embodiment. The dynamic balance estimation system 1 includes a gait measurement device 10 and a dynamic balance estimation device 13. In the present example embodiment, an example in which the gait measurement device 10 and the dynamic balance estimation device 13 are configured as separate hardware will be described. For example, the gait measurement device 10 is installed on footwear, or the like, of a subject (user) who is an estimation target of dynamic balance. For example, a function of the dynamic balance estimation device 13 is installed in a mobile terminal carried by the subject (user). Hereinafter, configurations of the gait measurement device 10 and the dynamic balance estimation device 13 will be individually described.

### [Gait Measurement Device]

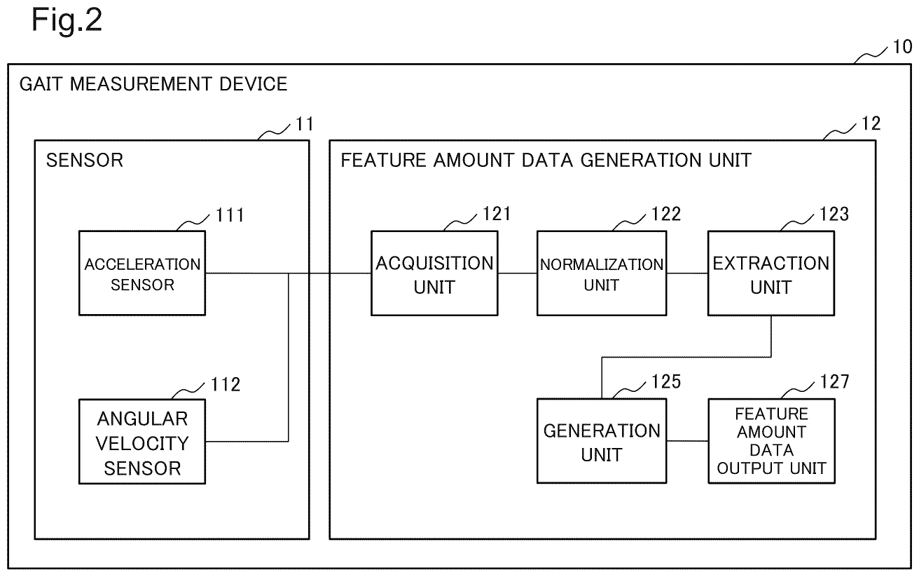

FIG. 2 is a block diagram illustrating an example of a configuration of the gait measurement device 10. The gait measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include sensors other than the acceleration sensor 111 and the angular velocity sensor 112. Description of the sensors other than the acceleration sensor 111 and the angular velocity sensor 112 that can be included in the sensor 11 will be omitted.

The acceleration sensor 111 is a sensor that measures acceleration (also referred to as spatial acceleration) in three-axial directions. The acceleration sensor 111 measures acceleration (also referred to as spatial acceleration) as a physical quantity regarding motion of the foot. The acceleration sensor 111 outputs the measured acceleration to the feature amount data generation unit 12. For example, a sensor of a piezoelectric type, a piezoresistive type, a capacitance type, or the like, can be used as the acceleration sensor 111. A measurement method of the sensor to be used as the acceleration sensor 111 is not limited as long as the sensor can measure acceleration.

The angular velocity sensor 112 is a sensor that measures angular velocity (also referred to as spatial angular velocity) around three axes. The angular velocity sensor 112 measures angular velocity (also referred to as spatial angular velocity) as a physical quantity regarding motion of the foot. The angular velocity sensor 112 outputs the measured angular velocity to the feature amount data generation unit 12. For example, a sensor of a vibration type, a capacitance type, or the like, can be used as the angular velocity sensor 112. A measurement method of the sensor to be used as the angular velocity sensor 112 is not limited as long as the sensor can measure angular velocity.

The sensor 11 is implemented by, for example, an inertial measurement device that measures acceleration and angular velocity. Examples of the inertial measurement device include an inertial measurement unit (IMU). The IMU includes the acceleration sensor 111 that measures acceleration in three-axial directions and the angular velocity sensor 112 that measures angular velocity around the three axes. The sensor 11 may be implemented by an inertial measurement device such as a vertical gyro (VG) or an attitude heading (AHRS). Furthermore, the sensor 11 may be implemented by a global positioning system/inertial navigation system (GPS/INS). The sensor 11 may be implemented by a device other than the inertial measurement device as long as it can measure a physical quantity regarding motion of the foot.

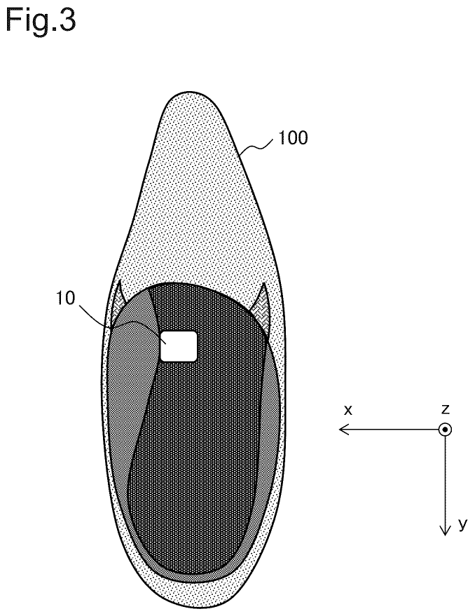

FIG. 3 is a conceptual diagram illustrating an example in which the gait measurement device 10 is arranged in a shoe 100 of the right foot. In the example of FIG. 3, the gait measurement device 10 is installed at a position corresponding to a back side of the arch of foot. For example, the gait measurement device 10 is arranged in an insole inserted into the shoe 100. For example, the gait measurement device 10 may be arranged on a bottom surface of the shoe 100. For example, the gait measurement device 10 may be embedded in the main body of the shoe 100. The gait measurement device 10 may be detachable from the shoe 100 or does not have to be detachable from the shoe 100. The gait measurement device 10 may be installed at a position other than the back side of the arch of foot as long as the sensor data regarding the motion of the foot can be measured. Furthermore, the gait measurement device 10 may be installed on a sock worn by the user or a decorative article such as an anklet worn by the user. Furthermore, the gait measurement device 10 may be directly attached to the foot or may be embedded in the foot. FIG. 3 illustrates an example in which the gait measurement device 10 is installed in the shoe 100 of the right foot. The gait measurement device 10 may be installed on the shoes 100 of both feet.

In the example of FIG. 3, a local coordinate system including an x axis in a left-right direction, a y axis in a front-back direction, and a z axis in an up-down direction is set based on the gait measurement device 10 (sensor 11). In the x-axis, a left side is positive, in the y-axis, a back side is positive, and in the z-axis, an upper side is positive. The directions of the axes set in the sensor 11 may be the same for the left and right feet, or may be different for the left and right feet. For example, in a case where the sensors 11 produced with the same specifications are arranged in the left and right shoes 100, up-down directions (directions in the Z-axis direction) of the sensors 11 arranged in the left and right shoes 100 are the same. In this case, three axes of the local coordinate system set in the sensor data derived from the left foot and three axes of the local coordinate system set in the sensor data derived from the right foot are the same on the left and right.

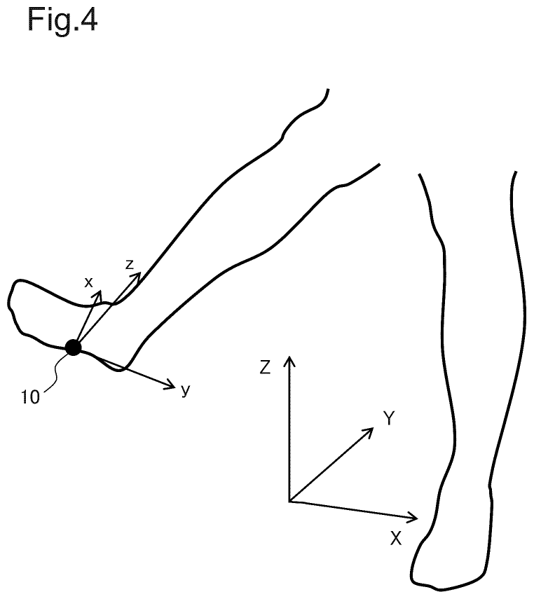

FIG. 4 is a conceptual diagram for explaining the local coordinate system (x-axis, y-axis, z-axis) set in the gait measurement device 10 (sensor 11) installed on the back side of the arch of foot and a world coordinate system (X axis, Y axis, Z axis) set with respect to the ground. In the world coordinate system (X axis, Y axis, Z axis), in a state where the user facing a traveling direction is upright, a lateral direction of the user is set to the X-axis direction (a leftward direction is positive), a direction of the back surface of the user is set to the Y-axis direction (a rearward direction is positive), and a gravity direction is set to the Z-axis direction (a vertically upward direction is positive). The example of FIG. 4 conceptually illustrates a relationship between the local coordinate system (x-axis, y-axis, z-axis) and the world coordinate system (X axis, Y axis, Z axis) and does not accurately illustrate a relationship between the local coordinate system and the world coordinate system that varies depending on gait of the user.

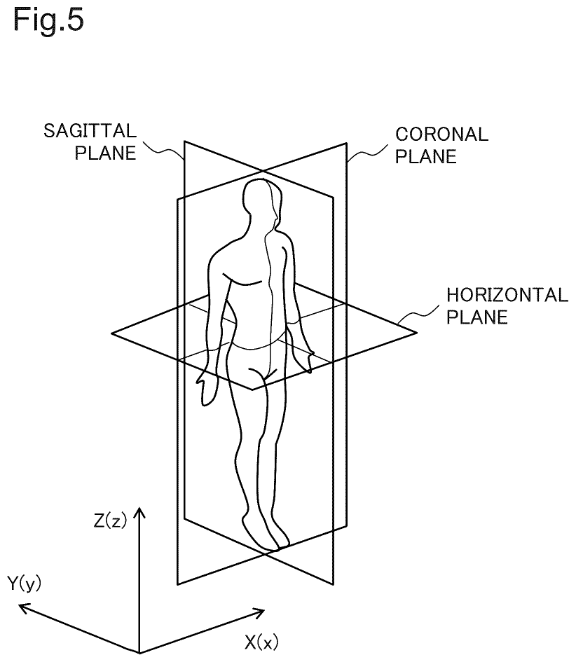

FIG. 5 is a conceptual diagram for explaining a surface (also referred to as a human body surface) set for the human body. In the present example embodiment, a sagittal plane dividing the body into left and right, a coronal plane dividing the body into front and back, and a horizontal plane dividing the body horizontally are defined. As illustrated in FIG. 5, the world coordinate system matches the local coordinate system in a state where the user is upright while the center line of the foot is oriented in the traveling direction. In the present example embodiment, rotation in the sagittal plane using the x axis as the rotation axis is defined as roll, rotation in the coronal plane using the y axis as the rotation axis is defined as pitch, and rotation in the horizontal plane using the z axis as the rotation axis is defined as yaw. In addition, a rotation angle in the sagittal plane using the x axis as the rotation axis is defined as a roll angle, a rotation angle in the coronal plane using the y axis as the rotation axis is defined as a pitch angle, and a rotation angle in the horizontal plane using the z axis as the rotation axis is defined as a yaw angle.

As illustrated in FIG. 2, the feature amount data generation unit 12 (also referred to as a feature amount data generation device) includes an acquisition unit 121, a normalization unit 122, an extraction unit 123, a generation unit 125, and a feature amount data output unit 127. For example, the feature amount data generation unit 12 is implemented by a microcomputer or a microcontroller that performs overall control and data processing of the gait measurement device 10. For example, the feature amount data generation unit 12 includes a central processing unit (CPU), a random access memory (RAM), a read only memory (ROM), a flash memory, and the like. The feature amount data generation unit 12 controls the acceleration sensor 111 and the angular velocity sensor 112 to measure the angular velocity and the acceleration. For example, the feature amount data generation unit 12 may be implemented on a mobile terminal (not illustrated) carried by the subject (user).

The acquisition unit 121 acquires acceleration in three-axial directions from the acceleration sensor 111. In addition, the acquisition unit 121 acquires angular velocity around three axes from the angular velocity sensor 112. For example, the acquisition unit 121 performs analog-to-digital conversion (AD conversion) on the acquired physical quantities (analog data) such as the angular velocity and the acceleration. The physical quantities (analog data) measured by the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in the three-axial directions. The angular velocity data includes angular velocity vectors around the three axes. The acceleration data and the angular velocity data are associated with time points at which the data is acquired. In addition, the acquisition unit 121 may add correction such as a mounting error, temperature correction, and linearity correction to the acceleration data and the angular velocity data.

The normalization unit 122 acquires the sensor data from the acquisition unit 121. The normalization unit 122 extracts time-series data (also referred to as gait waveform data) for one gait cycle from the time-series data of the acceleration in the three-axial directions and the angular velocity around the three axes included in the sensor data. The normalization unit 122 normalizes (also referred to as first normalization) a period of the extracted gait waveform data for one gait cycle to a gait cycle of 0 to 100% (percent). Timings such as 1% and 10% included in the gait cycle of 0 to 100% are also referred to as gait phases. Furthermore, the normalization unit 122 normalizes (also referred to as second normalization) the first normalized gait waveform data for one gait cycle in such a way that a stance phase becomes 60% and a swing phase becomes 40%. The stance phase is a period in which at least part of the back side of the foot is in contact with the ground. The swing phase is a period in which the back side of the foot is away from the ground. By performing second normalization on the gait waveform data, it is possible to prevent deviation of the gait phase from which the feature amount is extracted from fluctuating due to influence of disturbance.

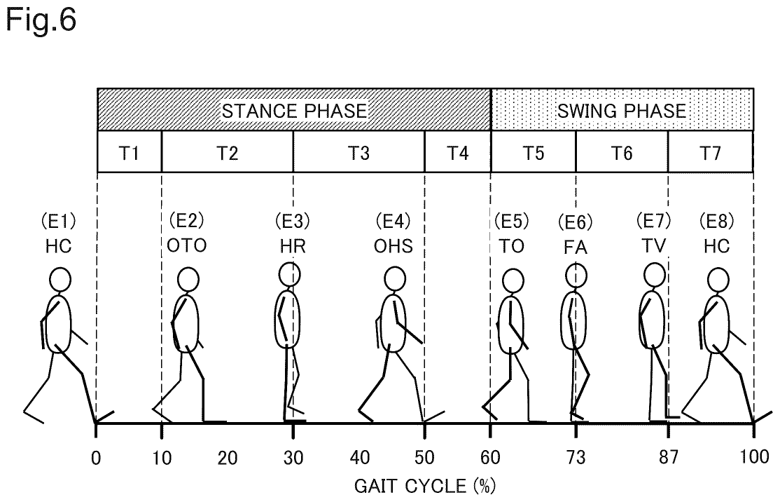

FIG. 6 is a conceptual diagram for explaining one gait cycle based on the right foot. One gait cycle based on the left foot is also similar to that of the right foot. The horizontal axis of FIG. 6 is one gait cycle of the right foot with a time point at which the heel of the right foot lands on the ground as a starting point and a time point at which the heel of the right foot next lands on the ground as an ending point. The horizontal axis in FIG. 6 is first normalized in such a way that one gait cycle becomes 100%. In addition, in the horizontal axis of FIG. 6, the second normalization is performed in such a way that the stance phase becomes 60% and the swing phase becomes 40%. The one gait cycle of one foot is roughly divided into a stance phase in which at least part of the back side of the foot is in contact with the ground and a swing phase in which the back side of the foot is away from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. FIG. 6 is an example and does not limit periods constituting one gait cycle, the names of these periods, and the like.

As illustrated in FIG. 6, in gait, a plurality of events (also referred to as gait events) occur. E1 represents an event in which the heel of the right foot comes into contact with the ground (heel contact) (heel contact (HC)). E2 represents an event in which the toe of the left foot is away from the ground (opposite toe off) in a state where the sole of the right foot is in contact with the ground (opposite toe off (OTO)). E3 represents an event in which the heel of the right foot lifts (heel rise) in a state where the sole of the right foot is in contact with the ground (heel rise (HR)). E4 is an event in which the heel of the left foot comes into contact with the ground (opposite heel strike) (opposite heel strike (OHS)). E5 represents an event in which the toe of the right foot is away from the ground (toe off) in a state where the sole of the left foot is in contact with the ground (toe off (TO)). E6 represents an event in which the left foot and the right foot cross (foot adjacent) in a state where the sole of the left foot is in contact with the ground (foot adjacent (FA)). E7 represents an event in which the tibia of the right foot is approximately perpendicular to the ground (tibia vertical) in a state where the sole of the left foot is in contact with the ground (tibia vertical (TV)). E8 represents an event in which the heel of the right foot comes into contact with the ground (heel contact) (heel contact (HC)). E8 is relevant to an end point of the gait cycle starting from E1 and is relevant to a start point of the next gait cycle. FIG. 6 is an example and does not limit events that occur during gait or names of these events.

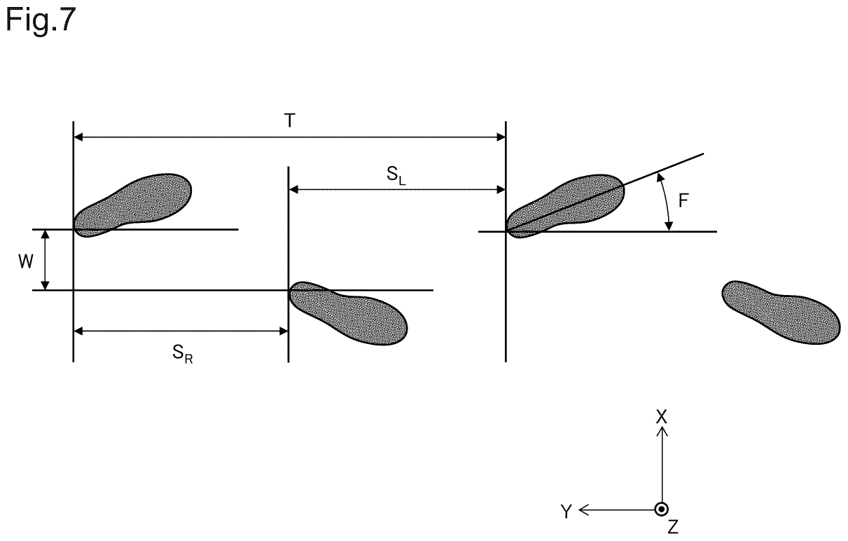

FIG. 7 is a conceptual diagram for explaining an example of gait parameters. FIG. 7 illustrates a right foot step length S_{R}, a left foot step length S_{L}, a stride length T, a step width W, and a foot angle F. The right foot step length S_{R} is a difference in the Y coordinate between the heel of the right foot and the heel of the left foot when the state in which the sole of the left foot is in contact with the ground transitions to the state in which the heel of the right foot swung out in the traveling direction lands on the ground. The left foot step length S_{L} is a difference in the Y coordinate between the heel of the left foot and the heel of the right foot when the state in which the sole of the right foot is in contact with the ground transitions to the state in which the heel of the left foot swung out in the traveling direction lands on the ground. The stride length T is a sum of the right foot step length S_{R} and the left foot step length S_{L}. The step width W is an interval between the right foot and the left foot. In FIG. 7, the step width W is a difference between the X coordinate of the center line of the heel of the right foot in a state where the right foot is in contact with the ground and the X coordinate of the center line of the heel of the left foot in a state where the left foot is in contact with the ground. The foot angle F is an angle formed by the center line of the foot and the traveling direction (Y axis) in a state where the back of the foot is in contact with the ground. In the present example embodiment, in the swing phase, the foot angle in a state where the foot floats in the air is evaluated.

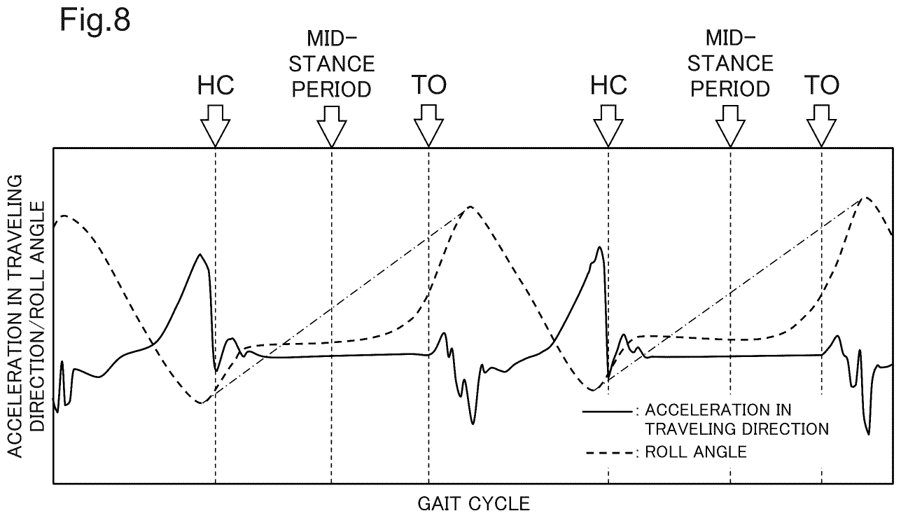

FIG. 8 is a view for explaining an example where the heel contact HC and the toe off TO are detected from the time-series data (solid line) of the acceleration in the traveling direction (acceleration in the Y direction). A timing of the heel contact HC is a timing of a minimum peak immediately after a maximum peak appearing in the time-series data of the acceleration in the traveling direction (acceleration in the Y direction). The maximum peak serving as a mark of the timing of the heel contact HC is relevant to a maximum peak of the gait waveform data for one gait cycle. An interval between the consecutive heel contacts HC is one gait cycle. A timing of the toe off TO is a rising timing of the maximum peak appearing after a period of the stance phase in which fluctuation does not appear in the time-series data of the acceleration in the traveling direction (acceleration in the Y direction). FIG. 8 also indicates time-series data (dashed line) of the roll angle (angular velocity around the X axis). A timing at the midpoint between the timing at which the roll angle is minimum and the timing at which the roll angle is maximum is relevant to the mid-stance period. For example, parameters (also referred to as gait parameters) such as gait speed, stride, minute, medial/lateral rotation, and plantarflexion/dorsiflexion can be obtained based on the mid-stance period.

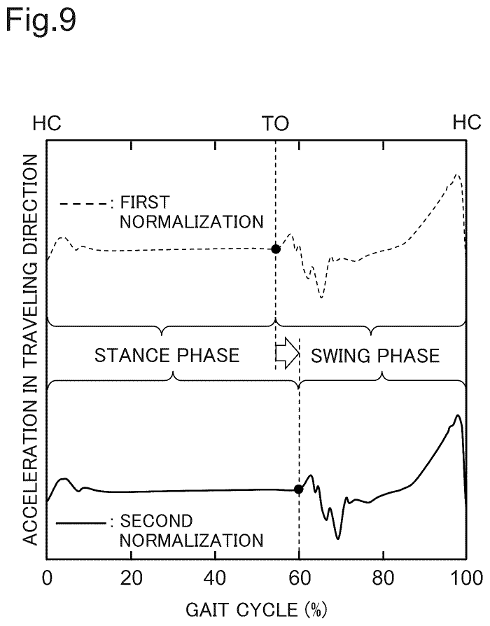

FIG. 9 is a view for explaining an example of the gait waveform data normalized by the normalization unit 122. The normalization unit 122 detects the heel contact HC and the toe off TO from the time-series data of the acceleration in the traveling direction (acceleration in the Y direction). The normalization unit 122 extracts an interval between consecutive heel contacts HC as the gait waveform data for one gait cycle. The normalization unit 122 converts the horizontal axis (time axis) of the gait waveform data for one gait cycle into a gait cycle of 0 to 100% by the first normalization. In FIG. 9, the gait waveform data after the first normalization is indicated by a dashed line. In the gait waveform data (dashed line) after the first normalization, the timing of the toe off TO is displaced from 60%.

In the example of FIG. 9, the normalization unit 122 normalizes an interval from the heel contact HC in which the gait phase is 0% to the toe off TO subsequent to the heel contact HC, to 0 to 60%. In addition, the normalization unit 122 normalizes an interval from the toe off TO to the heel contact HC in which the gait phase subsequent to the toe off TO is 100%, to 60 to 100%. As a result, the gait waveform data for one gait cycle is normalized to an interval (stance phase) in which the gait cycle is 0 to 60% and an interval (swing phase) in which the gait cycle is 60 to 100%. In FIG. 9, the gait waveform data after the second normalization is indicated by a solid line. In the gait waveform data (solid line) after the second normalization, the timing of the toe off TO coincides with 60%.

FIGS. 8 to 9 illustrate examples in which the gait waveform data for one gait cycle is extracted/normalized based on the acceleration in the traveling direction (acceleration in the Y direction). With respect to acceleration/angular velocity other than the acceleration in the traveling direction (acceleration in the Y direction), the normalization unit 122 extracts/normalizes the gait waveform data for one gait cycle in accordance with the gait cycle of the acceleration in the traveling direction (acceleration in the Y direction). Furthermore, the normalization unit 122 may generate time-series data of angles around three axes by integrating time-series data of the angular velocity around the three axes. In this case, the normalization unit 122 also extracts/normalizes the gait waveform data for one gait cycle in accordance with the gait cycle of the acceleration in the traveling direction (acceleration in the Y direction) with respect to the angles around the three axes.

The normalization unit 122 may extract/normalize the gait waveform data for one gait cycle based on acceleration/angular velocity other than the acceleration in the traveling direction (acceleration in the Y direction) (the drawings are omitted). For example, the normalization unit 122 may detect the heel contact HC and the toe off TO from the time-series data of acceleration in the vertical direction (acceleration in the Z direction). A timing of the heel contact HC is a timing of a steep minimum peak appearing in the time-series data of the acceleration in the vertical direction (acceleration in the Z direction). At the timing of the steep minimum peak, a value of the acceleration in the vertical direction (acceleration in the Z direction) becomes substantially zero. The minimum peak serving as a mark of the timing of the heel contact HC is relevant to the minimum peak of the gait waveform data for one gait cycle. An interval between the consecutive heel contacts HC is one gait cycle. A timing of the toe off TO is a timing of an inflection point in the process of the time-series data of the acceleration in the vertical direction (acceleration in the Z direction) gradually increasing after passing through an interval with small fluctuation after the maximum peak immediately after the heel contact HC. Furthermore, the normalization unit 122 may extract/normalize the gait waveform data for one gait cycle based on both the acceleration in the traveling direction (acceleration in the Y direction) and the acceleration in the vertical direction (acceleration in the Z direction). Furthermore, the normalization unit 122 may extract/normalize the gait waveform data for one gait cycle based on acceleration, angular velocity, angle, and the like, other than the acceleration in the traveling direction (acceleration in the Y direction) and the acceleration in the vertical direction (acceleration in the Z direction).

The extraction unit 123 acquires the gait waveform data for one gait cycle normalized by the normalization unit 122. The extraction unit 123 extracts a feature amount to be used for estimating the dynamic balance from the gait waveform data for one gait cycle. The extraction unit 123 extracts a feature amount for each gait phase cluster from a gait phase cluster obtained by integrating temporally consecutive gait phases based on conditions set in advance. The gait phase cluster includes at least one gait phase. The gait phase cluster also includes a single gait phase. The gait waveform data and the gait phase from which the feature amount to be used for estimating the dynamic balance is to be extracted will be described later.

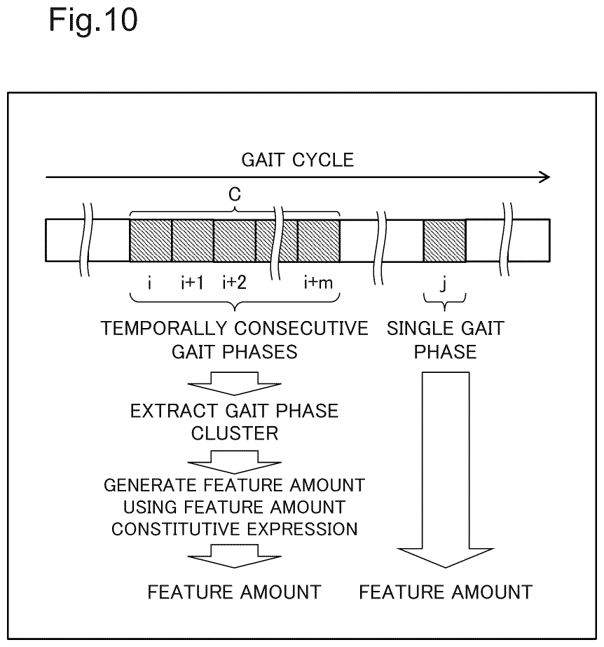

FIG. 10 is a conceptual diagram for explaining extraction of a feature amount for estimating the dynamic balance from the gait waveform data for one gait cycle. For example, the extraction unit 123 extracts temporally consecutive gait phases i to i+m as a gait phase cluster C (i and m are natural numbers). The gait phase cluster C includes m gait phases (components). In other words, the number of gait phases (components) (also referred to as the number of components) constituting the gait phase cluster C is m. FIG. 10 illustrates an example in which the gait phase has an integer value, but the gait phase may be subdivided into decimal places. In a case where the gait phase is subdivided into decimal places, the number of components of the gait phase cluster C is a number relevant to the number of data points in the interval of the gait phase cluster. The extraction unit 123 extracts a feature amount from each of the gait phases i to i+m. In a case where the gait phase cluster C includes a single gait phase j, the extraction unit 123 extracts a feature amount from the single gait phase j (j is a natural number).

The generation unit 125 applies a feature amount constitutive expression to the feature amount (first feature amount) extracted from each of the gait phases constituting the gait phase cluster to generate a feature amount (second feature amount) of the gait phase cluster. The feature amount constitutive expression is a calculation expression set in advance for generating the feature amount of the gait phase cluster. For example, the feature amount constitutive expression is a calculation expression regarding four arithmetic operations. For example, the second feature amount calculated using the feature amount constitutive equation is an integral average value, an arithmetic average value, a slope, a variation, or the like, of the first feature amount in each gait phase included in the gait phase cluster. For example, the generation unit 125 applies a calculation expression for calculating the slope and the variation of the first feature amount extracted from each of the gait phases constituting the gait phase cluster as the feature amount constitutive expression. For example, in a case where the gait phase cluster is constituted with a single gait phase, it is not possible to calculate the slope or the variation, and thus, it is only necessary t to use a feature amount constituent expression for calculating an integral average value, an arithmetic average value, or the like.

The feature amount data output unit 127 outputs the feature amount data for each gait phase cluster generated by the generation unit 125. The feature amount data output unit 127 outputs the generated feature amount data of the gait phase cluster to the dynamic balance estimation device 13 using the feature amount data.

### [Dynamic Balance Estimation Device]

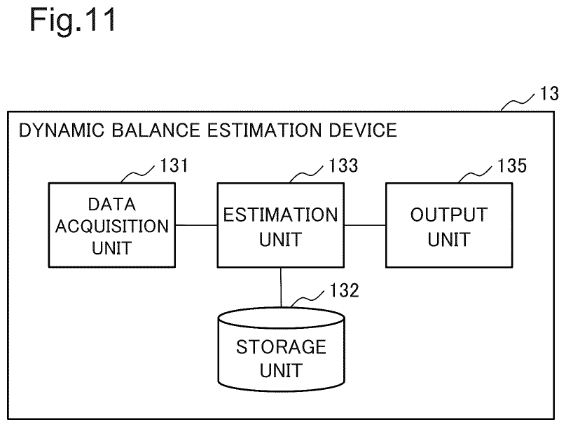

FIG. 11 is a block diagram illustrating an example of a configuration of the dynamic balance estimation device 13. The dynamic balance estimation device 13 includes a data acquisition unit 131, a storage unit 132, an estimation unit 133, and an output unit 135.

The data acquisition unit 131 acquires feature amount data from the gait measurement device 10. The data acquisition unit 131 outputs the received feature amount data to the estimation unit 133. The data acquisition unit 131 may receive the feature amount data from the gait measurement device 10 via a wire such as a cable or may receive the feature amount data from the gait measurement device 10 via wireless communication. For example, the data acquisition unit 131 is configured to receive the feature amount data from the gait measurement device 10 via a wireless communication function (not illustrated) conforming to standards such as Bluetooth (registered trademark) or WiFi (registered trademark). The communication function of the data acquisition unit 131 may conform to standards other than Bluetooth (registered trademark) or WiFi (registered trademark).

The storage unit 132 stores an estimation model for estimating the FR distance as the dynamic balance using the feature amount data extracted from the gait waveform data. The storage unit 132 stores an estimation model that has machine-learned a relationship between the feature amount data regarding the FR distances of the plurality of subjects and the FR distances. For example, the storage unit 132 stores an estimation model for estimating the FR distance, which has machine-learned for a plurality of subjects. The FR distance is affected by a height. Thus, the storage unit 132 may store an estimation model according to the attribute data regarding the height.

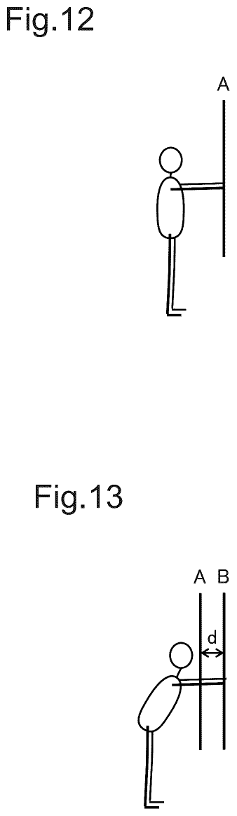

FIGS. 12 to 13 are conceptual diagrams for explaining the FR distance. FIG. 12 is a conceptual diagram illustrating a state in which both hands are raised by 90 degrees with respect to the horizontal plane. FIG. 12 illustrates a position A of fingertips of both raised hands. FIG. 13 is a conceptual diagram illustrating a state in which the upper limb is moved (reached) forward as much as possible from the state of FIG. 12. FIG. 13 illustrates the position A of the fingertips in the state of FIG. 12 and a position B of the fingertips in a state where the upper limb is moved forward as much as possible. A distance d between the position A and the position B is relevant to the FR distance.

The dynamic balance can be evaluated according to the value of the FR distance. In a case where the FR distance is equal to or longer than 30 cm (centimeters), the dynamic balance is high and a risk of falling is low. In a case where the FR distance falls in a range from 25 to 30 cm, the dynamic balance is average. In a case where the FR distance falls in a range from 20 to 25 cm, the dynamic balance is low, and there is a risk of falling. In a case where the FR distance is less than 20 cm, the dynamic balance is considerably low, and a risk of falling is very high. The evaluation criteria of the dynamic balance according to the FR distance described herein is a rough indication and may be set in accordance with the situation. For example, the evaluation criteria of the dynamic balance according to the value of the FR distance varies depending on the previous disease of the subject.

The estimation model may be stored in the storage unit 132 at the time of factory shipment of a product, calibration before the user uses the dynamic balance estimation system 1, or the like. For example, an estimation model stored in a storage device such as an external server may be used. In that case, the estimation model may be configured to be used via an interface (not illustrated) connected to the storage device.

The estimation unit 133 acquires the feature amount data from the data acquisition unit 131. The estimation unit 133 estimates the FR distance as the dynamic balance using the acquired feature amount data. The estimation unit 133 inputs the feature amount data to the estimation model stored in the storage unit 132. The estimation unit 133 outputs an estimation result according to the dynamic balance (FR distance) output from the estimation model. In a case where an estimation model stored in an external storage device constructed in a cloud, a server, or the like, is used, the estimation unit 133 is configured to use the estimation model via an interface (not illustrated) connected to the storage device.

The output unit 135 outputs the estimation result of the dynamic balance by the estimation unit 133. For example, the output unit 135 displays the estimation result of the dynamic balance on a screen of a mobile terminal of the subject (user). For example, the output unit 135 outputs the estimation result to an external system, or the like, that is to use the estimation result. The use of the dynamic balance output from the dynamic balance estimation device 13 is not particularly limited.

For example, the dynamic balance estimation device 13 is connected to an external system, or the like, constructed in a cloud or a server via a mobile terminal (not illustrated) carried by the subject (user). The mobile terminal (not illustrated) is a portable communication device. For example, the mobile terminal is a portable communication device having a communication function, such as a smartphone, a smart watch, or a mobile phone. For example, the dynamic balance estimation device 13 is connected to the mobile terminal via a wire such as a cable. For example, the dynamic balance estimation device 13 is connected to the mobile terminal via wireless communication. For example, the dynamic balance estimation device 13 is connected to the mobile terminal via a wireless communication function (not illustrated) conforming to standards such as Bluetooth (registered trademark) or WiFi (registered trademark). The communication function of the dynamic balance estimation device 13 may conform to standards other than Bluetooth (registered trademark) or WiFi (registered trademark). The estimation result of the dynamic balance may be used by an application installed on the mobile terminal. In that case, the mobile terminal executes processing using the estimation result by application software, or the like, installed in the mobile terminal.

### [FR Distance Estimation]

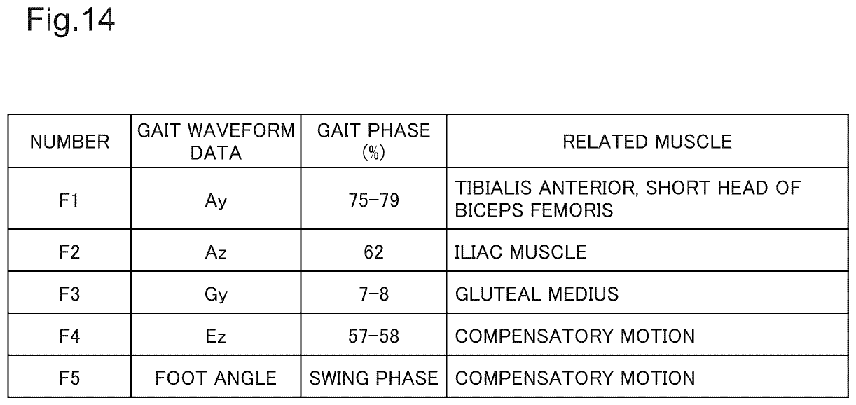

Next, a correlation between the FR distance and the feature amount data will be described with reference to a verification example. FIG. 14 is a correspondence table summarizing feature amounts to be used for estimating the FR distance. The correspondence table of FIG. 14 associates the number of the feature amount, the gait waveform data from which the feature amount is to be extracted, the gait phase (%) from which the gait phase cluster is to be extracted, and the related muscle. The FR distance is correlated with activities of the gluteus medius, the iliac muscles, the hamstrings (the long head of the biceps femoris), the tibialis anterior, and the like, and a magnitude of compensatory motion that makes orientation of the paw outward. Thus, the feature amounts F1 to F5 extracted from the gait phase in which these features appear are used to estimate the FR distance.

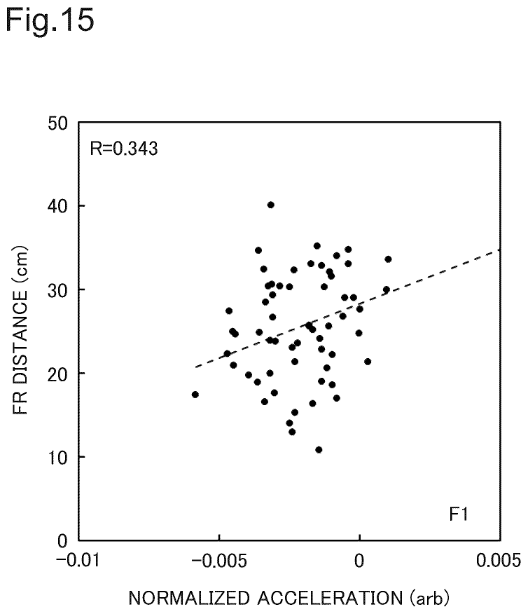

FIGS. 15 to 19 are verification results of the correlation between the FR distance and the feature amount data. FIGS. 15 to 19 indicate the results of verification performed on a total of 62 subjects including 27 males and 35 females aged 60 to 85 years. FIGS. 15 to 19 indicate the results of verifying a correlation between the estimated value estimated using the feature amount extracted in accordance with gait with the footwear on which the gait measurement device 10 is mounted and the measured value (true value) of the FR distance.

The feature amount F1 is extracted from an interval of the gait phase 75 to 79% of gait waveform data Ay related to the time-series data of the acceleration in the traveling direction (acceleration in the Y direction). The gait phase 75 to 79% is included in the mid-swing period T6. The feature amount F1 mainly includes a feature regarding motion of the tibialis anterior and the short head of the biceps femoris. FIG. 15 is a verification result of the correlation between the feature amount F1 and the FR distance. The horizontal axis of the graph of FIG. 15 indicates normalized acceleration. The correlation coefficient R between the feature amount F1 and the FR distance was 0.343.

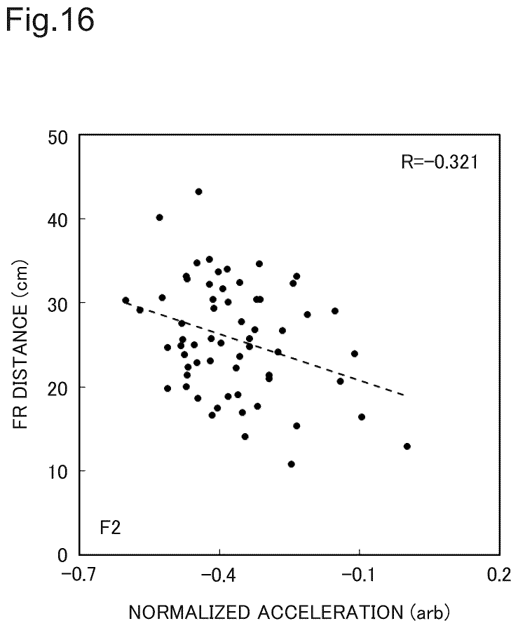

The feature amount F2 is extracted from an interval of the gait phase 62% of gait waveform data Az related to the time-series data of the acceleration in the vertical direction (acceleration in the Z direction). The gait phase 62% is included in the initial swing period T5. The feature amount F2 mainly includes a feature regarding motion of the iliac muscle. FIG. 16 is a verification result of the correlation between the feature amount F2 and the FR distance. The horizontal axis of the graph of FIG. 16 indicates normalized acceleration. The correlation coefficient R between the feature amount F2 and the FR distance was −0.321.

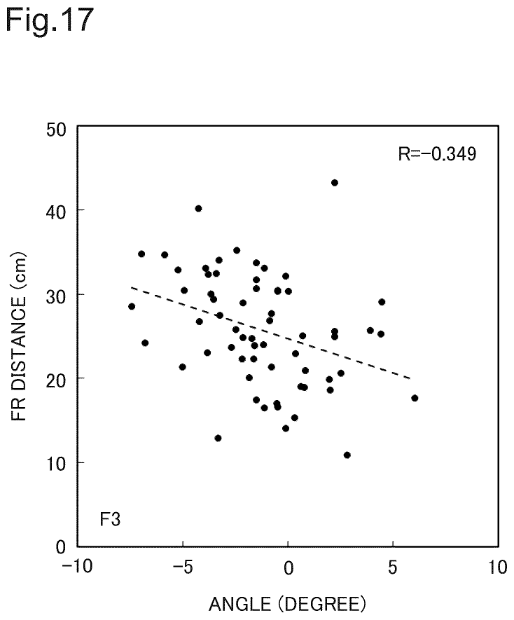

The feature amount F3 is extracted from an interval of the gait phase 7 to 8% of gait waveform data Gy related to the time-series data of the angular velocity in the coronal plane (around the Y axis). The gait phase 7 to 8% is included in the load response period T1. The feature amount F3 mainly includes a feature regarding motion of the gluteus medius. FIG. 17 is a verification result of the correlation between the feature amount F3 and the FR distance. The horizontal axis of the graph of FIG. 17 indicates an angle in the coronal plane. The correlation coefficient R between the feature amount F3 and the FR distance was −0.349.

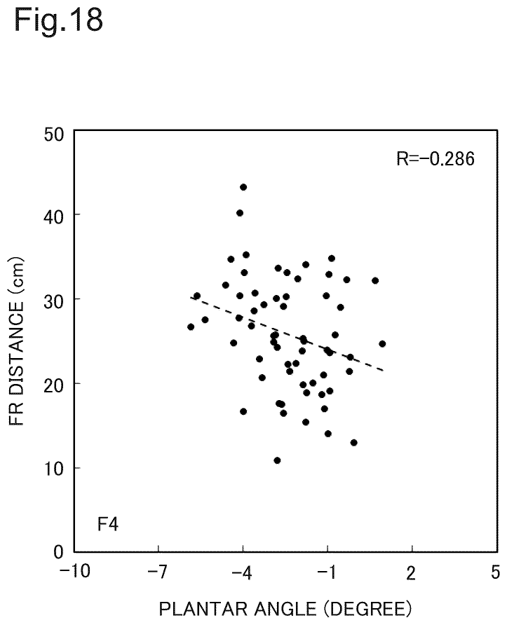

The feature amount F4 is extracted from an interval of the gait phase 57 to 58% of gait waveform data Ez related to the time-series data of an angle (posture angle) in the horizontal plane (around the Z axis). The gait phase 57 to 58% is included in the pre-swing period T4. The feature amount F4 mainly includes a feature related to compensatory motion. The compensatory motion is motion of changing the foot angle to acquire stability in order to compensate for decrease in the balance ability and muscle function associated with aging. FIG. 18 is a verification result of the correlation between the feature amount F4 and the FR distance. The horizontal axis of the graph in FIG. 18 indicates an angle (plantar angle) in the horizontal plane. The correlation coefficient R between the feature amount F4 and the FR distance was −0.286.

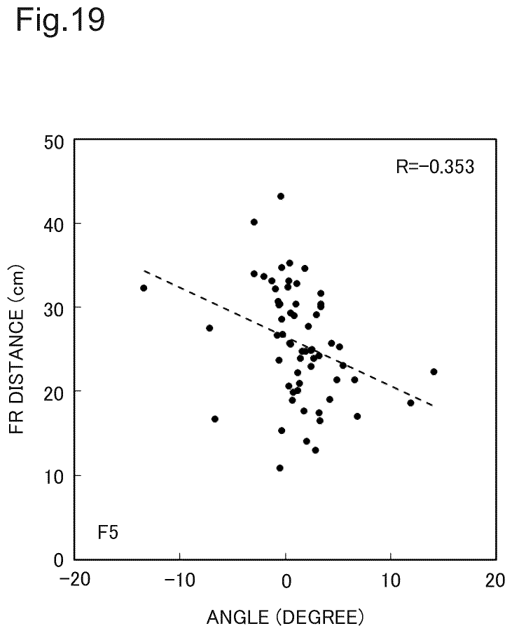

The feature amount F5 is an average value of the foot angles in the horizontal plane in the swing phase. For example, the feature amount F5 is an average value in the swing phase of the gait waveform data Ez. In other words, the feature amount F5 is an integral value of gait waveform data Gz related to the time-series data of the angular velocity in the horizontal plane (around the Z axis). The feature amount F5 mainly includes a feature regarding compensatory motion. The compensatory motion is motion of changing the foot angle to acquire stability in order to compensate for decrease in the balance ability and muscle function associated with aging. FIG. 19 is a verification result of the correlation between the feature amount F5 and the FR distance. The horizontal axis of the graph of FIG. 19 indicates an angle in the coronal plane. The correlation coefficient R between the feature amount F5 and the FR distance was −0.353.

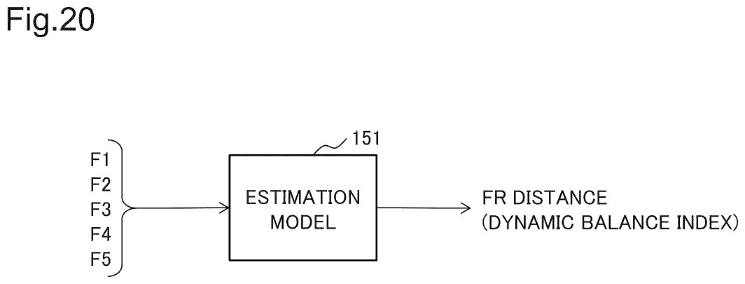

FIG. 20 is a conceptual diagram illustrating an example in which the estimated value of the FR distance is output by inputting the feature amounts F1 to F5 extracted from the sensor data measured in association with the gait of the user to the estimation model 151 constructed in advance to estimate the FR distance as the dynamic balance. The estimation model 151 outputs an FR distance which is an index of the dynamic balance in accordance with inputs of the feature amounts F1 to F5. For example, the estimation model 151 is generated by machine learning using teacher data having the feature amounts F1 to F5 to be used for estimating the FR distance as explanatory variables and the FR distance as an objective variable. The estimation result of the estimation model 151 is not limited as long as the estimation result regarding the FR distance, which is an index of the dynamic balance, is output in accordance with the input of the feature amount data for estimating the FR distance. For example, the estimation model 151 may be a model that estimates the FR distance using attribute data (height) as an explanatory variable in addition to the feature amounts F1 to F5 to be used for estimating the FR distance.

For example, the storage unit 132 stores an estimation model for estimating the FR distance using a multiple regression prediction method. For example, the storage unit 132 stores parameters for estimating the FR distance using the following expression 1.

FR distance=a1×F1+a2×F2+a3×F3+a4×F4+a5×F5+a0 (1)

In expression 1 described above, F1, F2, F3, F4, and F5 are feature amounts for each gait phase cluster to be used for estimating the FR distance indicated in the correspondence table of FIG. 14. a1, a2, a3, a4, and a5 are coefficients to be multiplied by F1, F2, F3, F4, and F5. a0 is a constant term. For example, a0, a1, a2, a3, a4, and a5 are stored in the storage unit 132.

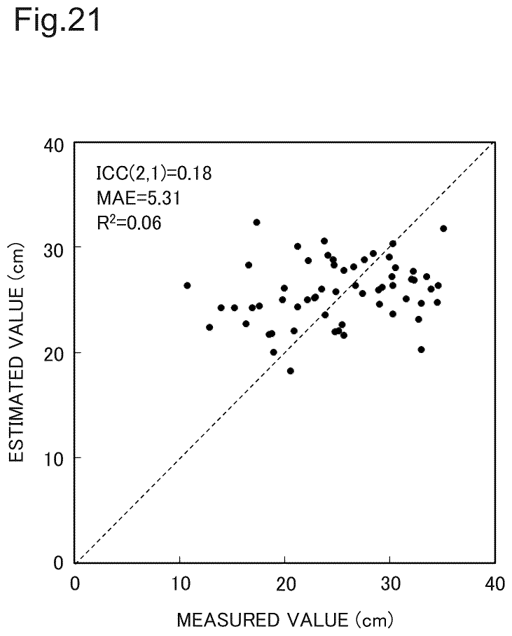

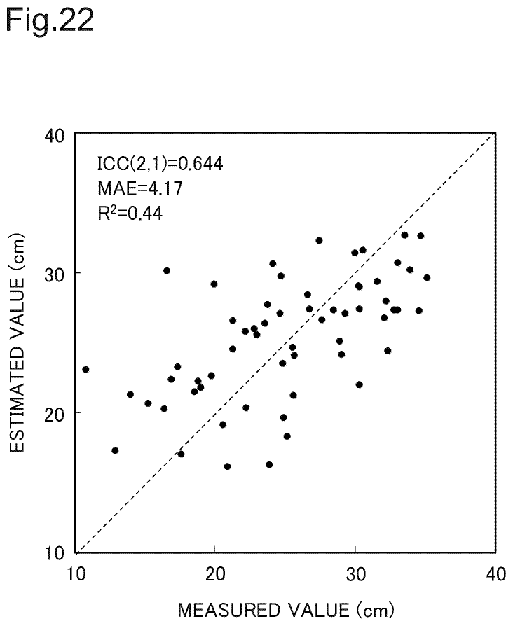

Next, a result of evaluating the estimation model 151 generated using the measurement data of 62 subjects described above will be described. Here, the verification example (FIG. 21) in which the dynamic balance (FR distance) is estimated using the attribute (including the gait speed) of the subject will be compared with the verification example (FIG. 22) in which the dynamic balance (FR distance) is estimated using the feature amount of the gait of the subject. FIGS. 21 and 22 indicate the results of testing the estimation model generated using the measurement data of 61 people using the measurement data of the remaining 1 person by a leave-one-subject-out (LOSO) method. FIGS. 21 and 22 indicate the results of performing LOSO on all (62) subjects and associating prediction values by the test with the measured values (true values). The test results of the LOSO were evaluated by values of intraclass correlation coefficient (ICC), a mean absolute error (MAE), and a determination coefficient R². As the intraclass correlation coefficient ICC, an intraclass correlation coefficient ICC (2, 1) was used in order to evaluate inter-examiner reliability.

FIG. 21 is a verification result of an estimation model of a comparative example in which teacher data having gender, age, height, weight, and gait speed as explanatory variables and an FR distance as an objective variable is machine-learned. In the estimation model of the comparative example, the intraclass correlation coefficient ICC (2, 1) was 0.18, the mean absolute error MAE was 5.31, and the determination coefficient R² was 0.06.

FIG. 22 indicates a verification result of the estimation model 151 of the present example embodiment machine-learned from teacher data having the feature amounts F1 to F5, the age, and the height as the explanatory variables and having the FR distance as the objective variable. In the estimation model 151 of the present example embodiment, the intraclass correlation coefficient ICC (2, 1) was 0.644, the mean absolute error MAE was 4.17, and the determination coefficient R² was 0.44. In other words, the estimation model 151 of the present example embodiment has higher reliability and smaller error than the estimation model of the comparative example, and the objective variable is sufficiently explained by the explanatory variables. In other words, according to the method of the present example embodiment, it is possible to generate the estimation model 151 that is highly reliable, has a small error, and has the objective variable sufficiently explained by the explanatory variables, as compared with the estimation model using only the attribute and the gait speed.

### (Operation)

Next, operation of the dynamic balance estimation system 1 will be described with reference to the drawings. Here, the gait measurement device 10 and the dynamic balance estimation device 13 included in the dynamic balance estimation system 1 will be individually described. Concerning the gait measurement device 10, operation of the feature amount data generation unit 12 included in the gait measurement device 10 will be described.

### [Gait Measurement Device]

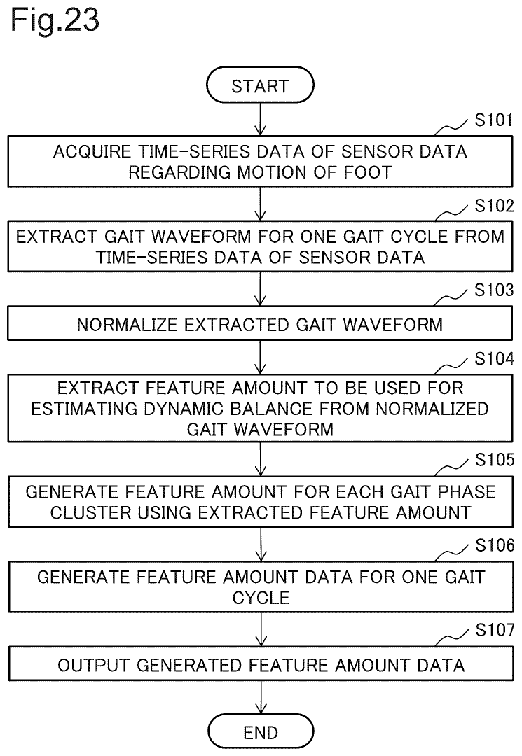

FIG. 23 is a flowchart for explaining operation of the feature amount data generation unit 12 included in the gait measurement device 10. In the explanation along the flowchart of FIG. 23, the feature amount data generation unit 12 will be described as an operation subject.

In FIG. 23, first, the feature amount data generation unit 12 acquires time-series data of sensor data regarding motion of the foot (step S101).

Next, the feature amount data generation unit 12 extracts gait waveform data for one gait cycle from the time-series data of the sensor data (step S102). The feature amount data generation unit 12 detects heel contact and toe off from the time-series data of the sensor data. The feature amount data generation unit 12 extracts time-series data of an interval between consecutive heel contacts as the gait waveform data for one gait cycle.

Next, the feature amount data generation unit 12 normalizes the extracted gait waveform data for one gait cycle (step S103). The feature amount data generation unit 12 normalizes the gait waveform data for one gait cycle to a gait cycle of 0 to 100% (first normalization). Furthermore, the feature amount data generation unit 12 normalizes a ratio of the stance phase to the swing phase in the first normalized gait waveform data for one gait cycle to 60:40 (second normalization).

Next, the feature amount data generation unit 12 extracts a feature amount from the gait phase to be used for estimating the dynamic balance concerning the normalized gait waveform (step S104). For example, the feature amount data generation unit 12 extracts a feature amount to be input to the estimation model constructed in advance.

Next, the feature amount data generation unit 12 generates a feature amount for each gait phase cluster using the extracted feature amount (step S105).

Next, the feature amount data generation unit 12 integrates the feature amounts for each gait phase cluster to generate feature amount data for one gait cycle (step S106).

Next, the feature amount data generation unit 12 outputs the generated feature amount data to the dynamic balance estimation device 13 (step S107).

[Dynamic Balance Estimation Device]

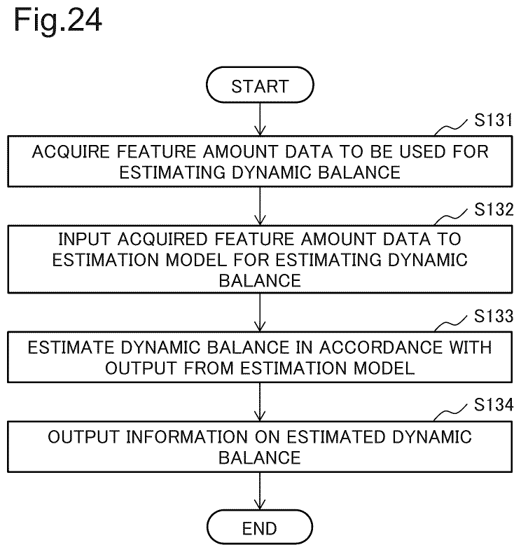

FIG. 24 is a flowchart for explaining operation of the dynamic balance estimation device 13. In the explanation along the flowchart of FIG. 24, the dynamic balance estimation device 13 will be described as an operation subject.

In FIG. 24, first, the dynamic balance estimation device 13 acquires the feature amount data generated using the sensor data regarding motion of the foot (step S131).

Next, the dynamic balance estimation device 13 inputs the acquired feature amount data to the estimation model for estimating the dynamic balance (FR distance) (step S132).

Next, the dynamic balance estimation device 13 estimates the dynamic balance of the user in accordance with the output (estimated value) from the estimation model (step S133). For example, the dynamic balance estimation device 13 estimates the FR distance of the user as the dynamic balance.

Next, the dynamic balance estimation device 13 outputs information on the estimated dynamic balance (step S134). For example, the dynamic balance is output to a terminal device (not illustrated) carried by the user. For example, the dynamic balance is output to a system that executes processing using the dynamic balance.

### (Application Example)

Next, an application example according to the present example embodiment will be described with reference to the drawings. In the following application example, an example in which the function of the dynamic balance estimation device 13 installed in the mobile terminal carried by the user estimates the information on the dynamic balance using the feature amount data measured by the gait measurement device arranged in the shoe will be described.

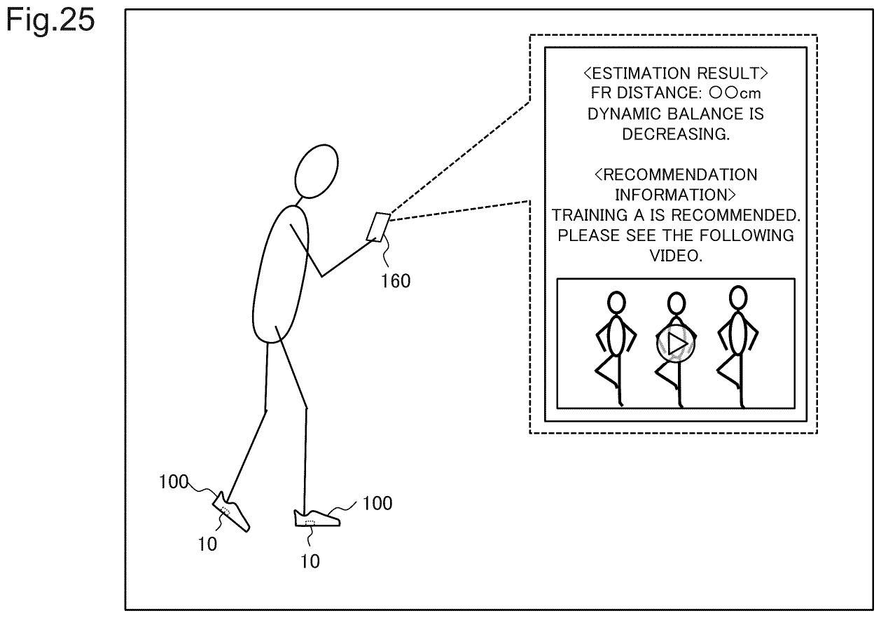

FIG. 25 is a conceptual diagram illustrating an example in which the estimation result by the dynamic balance estimation device 13 is displayed on a screen of the mobile terminal 160 carried by the user during gait while wearing the shoes 100 on which the gait measurement device 10 is arranged. FIG. 25 is an example in which information according to the estimation result of the dynamic balance using the feature amount data according to the sensor data measured during gait of the user is displayed on the screen of the mobile terminal 160.

FIG. 25 illustrates an example in which information corresponding to the estimated value of the FR distance that is the dynamic balance is displayed on the screen of the mobile terminal 160. In the example of FIG. 25, the estimated value of the FR distance is displayed on a display unit of the mobile terminal 160 as the estimation result of the dynamic balance. Furthermore, in the example of FIG. 25, information on the estimation result of the dynamic balance of “The dynamic balance is decreasing.” is displayed on the display unit of the mobile terminal 160 in accordance with the estimated value of the FR distance that is the dynamic balance. Furthermore, in the example of FIG. 25, recommendation information according to the estimation result of the dynamic balance like “Training A is recommended. Please see the following video.” is displayed on the display unit of the mobile terminal 160 in accordance with the estimated value of the FR distance that is the dynamic balance. The user who has confirmed the information displayed on the display unit of the mobile terminal 160 can practice training that leads to increase in the dynamic balance by exercising with reference to the video of the training A in accordance with the recommendation information.

As described above, the dynamic balance estimation system of the present example embodiment includes the gait measurement device and the dynamic balance estimation device. The gait measurement device includes the sensor and the feature amount data generation unit. The sensor includes the acceleration sensor and the angular velocity sensor. The sensor measures spatial acceleration using the acceleration sensor. The sensor measures spatial angular velocity using the angular velocity sensor. The sensor generates sensor data regarding motion of the foot using the measured spatial acceleration and spatial angular velocity. The sensor outputs the generated sensor data to the feature amount data generation unit. The feature amount data generation unit acquires time-series data of the sensor data regarding motion of the foot. The feature amount data generation unit extracts gait waveform data for one gait cycle from the time-series data of the sensor data. The feature amount data generation unit normalizes the extracted gait waveform data. The feature amount data generation unit extracts, from the normalized gait waveform data, a feature amount to be used for estimating the dynamic balance from a gait phase cluster constituted with at least one temporally consecutive gait phase. The feature amount data generation unit generates feature amount data including the extracted feature amount. The feature amount data generation unit outputs the generated feature amount data.

The dynamic balance estimation device includes a data acquisition unit, a storage unit, an estimation unit, and an output unit. The data acquisition unit acquires the feature amount data including the feature amount to be used for estimating the dynamic balance of the user extracted from the sensor data regarding motion of the foot of the user. The storage unit stores the estimation model that outputs the dynamic balance index according to the input of the feature amount data. The estimation unit inputs the acquired feature amount data to the estimation model. The estimation unit estimates the dynamic balance of the user in accordance with the dynamic balance index output from the estimation model. The output unit outputs information on the estimated dynamic balance.

The dynamic balance estimation system of the present example embodiment estimates the dynamic balance of the user using the feature amount extracted from the sensor data regarding the motion of the foot of the user. Thus, according to the dynamic balance estimation system of the present example embodiment, the dynamic balance can be appropriately estimated in daily life without using an instrument for measuring the dynamic balance.

In one aspect of the present example embodiment, the data acquisition unit acquires the feature amount data including the feature amount extracted from the gait waveform data generated using the time-series data of the sensor data regarding the motion of the foot. The data acquisition unit acquires feature amount data including a feature amount to be used for estimating a value of a result (functional reach distance) of a functional reach test as the dynamic balance index. According to the present aspect, by using the sensor data regarding the motion of the foot, the dynamic balance can be appropriately estimated in daily life without using an instrument for measuring the dynamic balance.

In one aspect of the present example embodiment, the storage unit stores an estimation model generated by machine learning using teacher data regarding a plurality of subjects. The estimation model is generated by machine learning using teacher data having a feature amount to be used for estimating the dynamic balance index as the explanatory variable and dynamic balance indexes of a plurality of subjects as objective variables. The estimation unit inputs the feature amount data acquired regarding the user to the estimation model. The estimation unit estimates the dynamic balance of the user in accordance with the dynamic balance index of the user output from the estimation model. According to the present aspect, the dynamic balance can be appropriately estimated in daily life without using an instrument for measuring the dynamic balance.

In one aspect of the present example embodiment, the storage unit stores the estimation model machine-learned using the explanatory variables including the attribute data (height) of the subject. The estimation unit inputs the feature amount data and the attribute data (height) regarding the user to the estimation model. The estimation unit estimates the dynamic balance of the user in accordance with the dynamic balance index of the user output from the estimation model. In the present aspect, the dynamic balance is estimated including the attribute data (height) that affects the dynamic balance. Thus, according to the present aspect, the dynamic balance can be measured with higher accuracy.

In one aspect of the present example embodiment, the storage unit stores an estimation model generated by machine learning using teacher data regarding a plurality of subjects. The estimation model is a model generated by machine learning using teacher data having feature amounts extracted from the gait waveform data of the plurality of subjects as explanatory variables and dynamic balance indexes of the plurality of subjects as objective variables. For example, the explanatory variables include a feature amount regarding an activity of the gluteus medius extracted from the load response period. For example, the explanatory variables include a feature amount regarding an activity of the iliac muscle extracted from the initial swing period. For example, the explanatory variables include a feature amount regarding the tibialis anterior and the short head of biceps femoris muscle extracted from the mid-swing period, and a feature amount regarding the compensatory motion of the foot angle in the swing phase. The estimation unit inputs the feature amount data acquired in accordance with gait of the user to the estimation model. The estimation unit estimates the dynamic balance of the user in accordance with the dynamic balance index of the user output from the estimation model. According to the present aspect, the dynamic balance more suitable for the physical activity can be estimated by using the estimation model that has machine-learned the feature amounts according to the activities of the muscles that affect the dynamic balance.

In one aspect of the present example embodiment, the storage unit stores, for a plurality of subjects, an estimation model generated by machine learning using teacher data having a plurality of feature amounts extracted from gait waveform data as explanatory variables and dynamic balance regarding dynamic balance indexes of the subjects as an objective variable. For example, the explanatory variables include a feature amount extracted from the load response period of the gait waveform data of the angular velocity in the coronal plane. For example, the explanatory variables include a feature amount extracted from the initial swing period of the gait waveform data of the acceleration in the vertical direction. For example, the explanatory variables include a feature amount extracted from the mid-swing period of the gait waveform data of the acceleration in the traveling direction. For example, the explanatory variables include a feature amount extracted from the pre-swing period of the gait waveform data of the angle in the horizontal plane. For example, the explanatory variables include a feature amount related to the foot angle in the swing phase. The data acquisition unit acquires the feature amount data including the feature amount extracted in accordance with gait of the user. For example, the data acquisition unit acquires the feature amount in the load response period of the gait waveform data of the angular velocity in the coronal plane. For example, the data acquisition unit acquires the feature amount of the initial swing period of the gait waveform data of the acceleration in the vertical direction. For example, the data acquisition unit acquires the feature amount of the mid-swing period of the gait waveform data of the acceleration in the traveling direction. For example, the data acquisition unit acquires the feature amount of the pre-swing period of the gait waveform data of the angle in the horizontal plane. For example, the data acquisition unit acquires the feature amount of the foot angle in the swing phase. The estimation unit inputs the acquired feature amount data to the estimation model. The estimation unit estimates the dynamic balance of the user in accordance with the dynamic balance index of the user output from the estimation model. According to the present aspect, by using the estimation model that has machine-learned the feature amount extracted from the gait waveform data including the feature according to the activities of the muscles that affect the dynamic balance, the dynamic balance more suitable for the physical activity can be estimated using the sensor data regarding the motion of the foot.

In one aspect of the present example embodiment, the dynamic balance estimation device is implemented in a terminal device having a screen visually recognizable by the user. For example, the dynamic balance estimation device displays information on the dynamic balance estimated in accordance with the motion of the foot of the user on the screen of the terminal device. For example, the dynamic balance estimation device displays recommendation information according to the dynamic balance estimated in accordance with the motion of the foot of the user on the screen of the terminal device. For example, the dynamic balance estimation device displays a video related to training for training a body part related to the dynamic balance on the screen of the terminal device as the recommendation information according to the dynamic balance estimated in accordance with the motion of the foot of the user. According to the present aspect, the dynamic balance estimated in accordance with the feature amount extracted from the sensor data regarding the motion of the foot of the user is displayed on the screen visually recognizable by the user, so that the user can confirm the information according to the dynamic balance of the user.

### Second Example Embodiment

Next, a machine learning system according to a second example embodiment will be described with reference to the drawings. The machine learning system of the present example embodiment generates an estimation model for estimating the dynamic balance according to the input of the feature amount by machine learning using the feature amount data extracted from the sensor data measured by the gait measurement device.

### (Configuration)

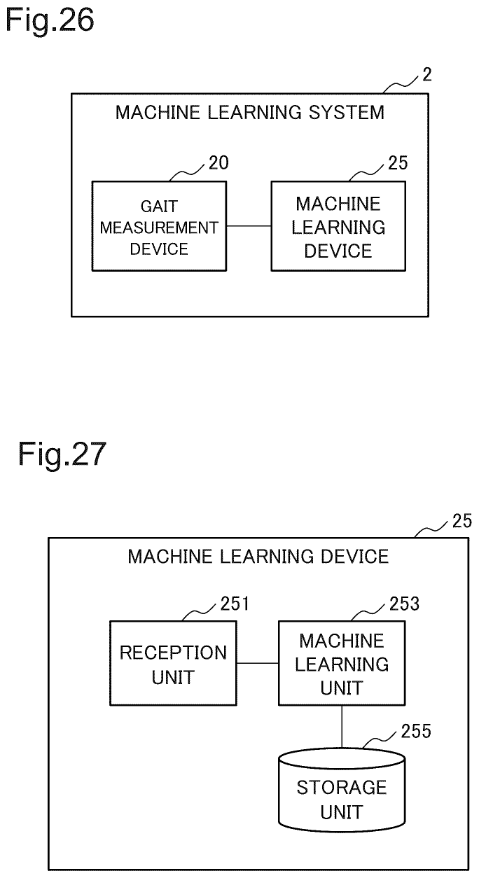

FIG. 26 is a block diagram illustrating an example of a configuration of a machine learning system 2 according to the present example embodiment. The machine learning system 2 includes a gait measurement device 20 and a machine learning device 25. The gait measurement device 20 and the machine learning device 25 may be connected in a wired or wireless manner. The gait measurement device 20 and the machine learning device 25 may be constituted with a single device. In addition, the machine learning system 2 may be constituted only with the machine learning device 25 by removing the gait measurement device 20 from the configuration of the machine learning system 2. Although only one gait measurement device 20 is illustrated in FIG. 26, one (two in total) gait measurement device 20 may be arranged on each of the left and right feet. Furthermore, the machine learning device 25 may be configured not to be connected to the gait measurement device 20 but to execute machine learning using the feature amount data generated in advance by the gait measurement device 20 and stored in a database.

The gait measurement device 20 is installed on at least one of the left and right feet. The gait measurement device 20 has the same configuration as the gait measurement device 10 of the first example embodiment. The gait measurement device 20 includes an acceleration sensor and an angular velocity sensor. The gait measurement device 20 converts the measured physical quantity into digital data (also referred to as sensor data). The gait measurement device 20 generates normalized gait waveform data for one gait cycle from the time-series data of the sensor data. The gait measurement device 20 generates feature amount data to be used for estimating the dynamic balance. The gait measurement device 20 transmits the generated feature amount data to the machine learning device 25. The gait measurement device 20 may be configured to transmit the feature amount data to a database (not illustrated) to be accessed by the machine learning device 25. The feature amount data accumulated in the database is used for machine learning by the machine learning device 25.

The machine learning device 25 receives the feature amount data from the gait measurement device 20. In a case where the feature amount data accumulated in the database (not illustrated) is used, the machine learning device 25 receives the feature amount data from the database. The machine learning device 25 executes machine learning using the received feature amount data. For example, the machine learning device 25 machine learns teacher data having pieces of the feature amount data extracted from a plurality of pieces of subject gait waveform data as explanatory variables and values related to the dynamic balance according to the feature amount data as objective variables. A machine learning algorithm to be executed by the machine learning device 25 is not particularly limited. The machine learning device 25 generates an estimation model machine-learned using teacher data regarding a plurality of subjects. The machine learning device 25 stores the generated estimation model. The estimation model machine-learned by the machine learning device 25 may be stored in a storage device outside the machine learning device 25.

### [Machine Learning Device]

Next, details of the machine learning device 25 will be described with reference to the drawings. FIG. 27 is a block diagram illustrating an example of a detailed configuration of the machine learning device 25. The machine learning device 25 includes a reception unit 251, a machine learning unit 253, and a storage unit 255.

The reception unit 251 receives the feature amount data from the gait measurement device 20. The reception unit 251 outputs the received feature amount data to the machine learning unit 253. The reception unit 251 may receive the feature amount data from the gait measurement device 20 via a wire such as a cable or may receive the feature amount data from the gait measurement device 20 via wireless communication. For example, the reception unit 251 is configured to receive the feature amount data from the gait measurement device 20 via a wireless communication function (not illustrated) conforming to standards such as Bluetooth (registered trademark) or WiFi (registered trademark). The communication function of the reception unit 251 may conform to standards other than Bluetooth (registered trademark) or WiFi (registered trademark).

The machine learning unit 253 acquires the feature amount data from the reception unit 251. The machine learning unit 253 executes machine learning using the acquired feature amount data. For example, the machine learning unit 253 performs machine learning a data set as teacher data, the data set having the feature amount data extracted from the sensor data measured in accordance with the motion of the foot of the subject as an explanatory variable and the FR distance of the subject as an objective variable. For example, the machine learning unit 253 generates an estimation model which is machine-learned for a plurality of subjects and which estimates the FR distance in accordance with the input of the feature amount data. For example, the machine learning unit 253 generates the estimation model according to the attribute data (height). For example, the machine learning unit 253 generates an estimation model that estimates the FR distance as the dynamic balance using the feature amount data extracted from the sensor data measured in accordance with the motion of the foot of the subject and the attribute data (height) of the subject as explanatory variables. The machine learning unit 253 stores estimation models machine-learned for a plurality of subjects in the storage unit 255.

For example, the machine learning unit 253 executes machine learning using a linear regression algorithm. For example, the machine learning unit 253 executes machine learning using an algorithm of a support vector machine (SVM). For example, the machine learning unit 253 executes machine learning using a Gaussian process regression (GPR) algorithm. For example, the machine learning unit 253 executes machine learning using a random forest (RF) algorithm. For example, the machine learning unit 253 may execute unsupervised machine learning of classifying a subject who is a generation source of the feature amount data in accordance with the feature amount data. The machine learning algorithm to be executed by the machine learning unit 253 is not particularly limited.

The machine learning unit 253 may execute machine learning using the gait waveform data for one gait cycle as an explanatory variable. For example, the machine learning unit 253 executes supervised machine learning using acceleration in the three-axial directions, angular velocity around the three axes, and gait waveform data of angles (posture angles) around the three axes as explanatory variables and a correct value of the dynamic balance index as an objective variable. For example, in a case where the gait phases are set in increments of 1% in a gait cycle of 0 to 100%, the machine learning unit 253 performs machine learning using 909 explanatory variables.

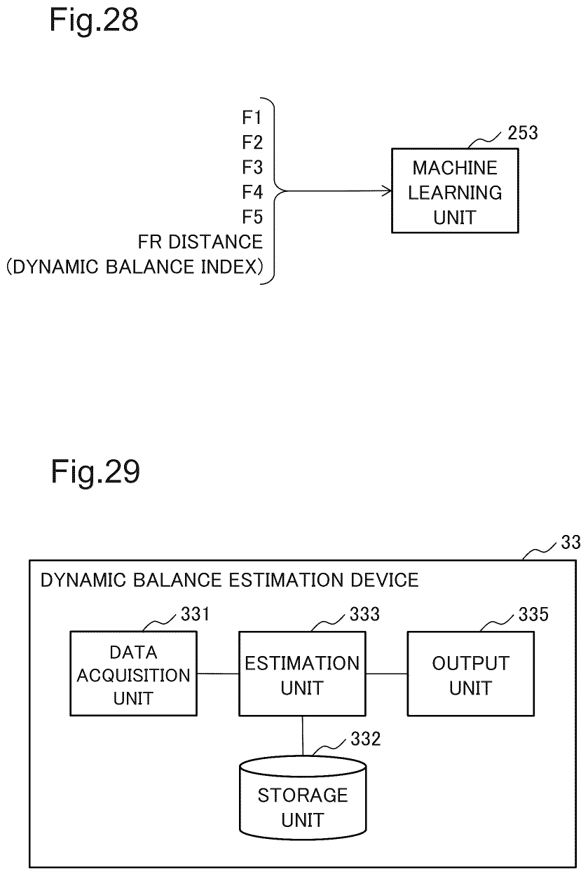

FIG. 28 is a conceptual diagram for explaining machine learning for generating an estimation model. FIG. 28 is a conceptual diagram illustrating an example of causing the machine learning unit 253 to perform machine learning using a data set of the feature amounts F1 to F5 that are explanatory variables and the FR distance (dynamic balance index) that is an objective variable as teacher data. For example, the machine learning unit 253 machine learns data regarding a plurality of subjects and generates an estimation model that outputs an output (estimated value) related to an FR distance (dynamic balance index) in accordance with the input of the feature amount extracted from the sensor data.

The storage unit 255 stores estimation models machine-learned for a plurality of subjects. For example, the storage unit 255 stores an estimation model for estimating the dynamic balance, machine-learned for a plurality of subjects. For example, the estimation model stored in the storage unit 255 is used for estimating the dynamic balance by the dynamic balance estimation device 13 of the first example embodiment.

As described above, the machine learning system of the present example embodiment includes the gait measurement device and the machine learning device. The gait measurement device acquires time-series data of the sensor data regarding motion of the foot. The gait measurement device extracts gait waveform data for one gait cycle from the time-series data of the sensor data and normalizes the extracted gait waveform data. The gait measurement device extracts, from the normalized gait waveform data, a feature amount to be used for estimating the dynamic balance of the user from a gait phase cluster constituted with at least one temporally consecutive gait phase. The gait measurement device generates feature amount data including the extracted feature amount. The gait measurement device outputs the generated feature amount data to the machine learning device.

The machine learning device includes a reception unit, a machine learning unit, and a storage unit. The reception unit acquires the feature amount data generated by the gait measurement device. The machine learning unit executes machine learning using the feature amount data. The machine learning unit generates the estimation model that outputs the dynamic balance in accordance with the input of the feature amount (second feature amount) of the gait phase cluster extracted from the time-series data of the sensor data measured in association with gait of the user. The estimation model generated by the machine learning unit is stored in the storage unit.

The machine learning system of the present example embodiment generates an estimation model using the feature amount data measured by the gait measurement device. Thus, according to the present aspect, it is possible to generate an estimation model capable of appropriately estimating the dynamic balance in daily life without using an instrument for measuring the dynamic balance.

### Third Example Embodiment

Next, a dynamic balance estimation device according to a third example embodiment will be described with reference to the drawings. The dynamic balance estimation device of the present example embodiment has a simplified configuration of the dynamic balance estimation device included in the dynamic balance estimation system of the first example embodiment.

FIG. 29 is a block diagram illustrating an example of a configuration of a dynamic balance estimation device 33 according to the present example embodiment. The dynamic balance estimation device 33 includes a data acquisition unit 331, a storage unit 332, an estimation unit 333, and an output unit 335.

The data acquisition unit 331 acquires feature amount data including a feature amount to be used for estimating the dynamic balance index of the user, extracted from sensor data regarding motion of the foot of the user. The storage unit 332 stores an estimation model that outputs the dynamic balance index according to the input of the feature amount data. The estimation unit 333 inputs the acquired feature amount data to the estimation model to estimate the dynamic balance of the user in accordance with the dynamic balance index output from the estimation model. The output unit 335 outputs information on the estimated dynamic balance.

As described above, in the present example embodiment, the dynamic balance of the user is estimated using the feature amount extracted from the sensor data regarding the motion of the foot of the user. Thus, according to the present example embodiment, the dynamic balance can be appropriately estimated in daily life without using an instrument for measuring the dynamic balance.

### (Hardware)

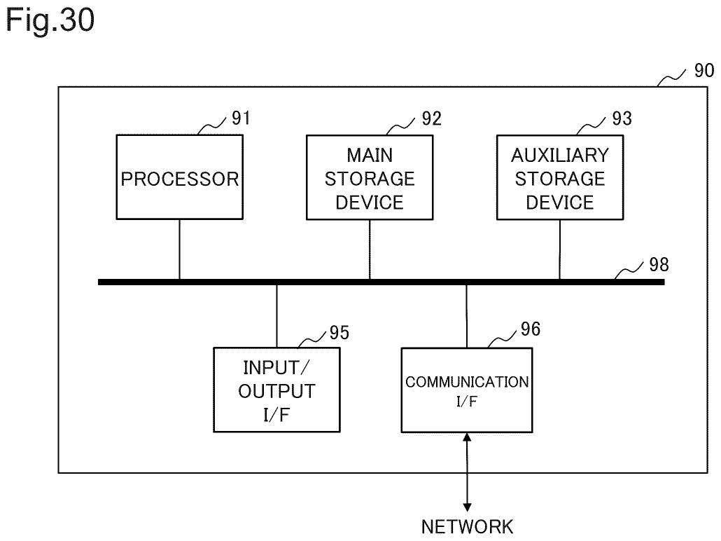

Here, a hardware configuration for executing control and processing according to each example embodiment of the present disclosure will be described using an information processing apparatus 90 in FIG. 30 as an example. The information processing apparatus 90 in FIG. 30 is a configuration example for executing control and processing of each example embodiment and does not limit the scope of the present disclosure.

As illustrated in FIG. 30, the information processing apparatus 90 includes a processor 91, a main storage device 92, an auxiliary storage device 93, an input/output interface 95, and a communication interface 96. In FIG. 30, the interface is abbreviated as an interface (I/F). The processor 91, the main storage device 92, the auxiliary storage device 93, the input/output interface 95, and the communication interface 96 are connected to each other via a bus 98 in such a way as to be able to perform data communication. In addition, the processor 91, the main storage device 92, the auxiliary storage device 93, and the input/output interface 95 are connected to a network such as the Internet or an intranet via the communication interface 96.

The processor 91 loads the program stored in the auxiliary storage device 93, or the like, in the main storage device 92. The processor 91 executes the program loaded in the main storage device 92. In the present example embodiment, it is only necessary to use a software program installed in the information processing apparatus 90. The processor 91 executes control and processing according to each example embodiment.

The main storage device 92 has an area in which a program is to be loaded. A program stored in the auxiliary storage device 93, or the like, is loaded in the main storage device 92 by the processor 91. The main storage device 92 is implemented by, for example, a volatile memory such as a dynamic random access memory (DRAM). In addition, a non-volatile memory such as a magnetoresistive random access memory (MRAM) may be constituted/added as the main storage device 92.

The auxiliary storage device 93 stores various kinds of data such as programs. The auxiliary storage device 93 is implemented by a local disk such as a hard disk or a flash memory. Various kinds of data may be stored in the main storage device 92, and the auxiliary storage device 93 may be omitted.

The input/output interface 95 is an interface for connecting the information processing apparatus 90 and a peripheral device in accordance with standards or specifications. The communication interface 96 is an interface for connecting to an external system or device through a network such as the Internet or an intranet in accordance with standards or specifications. The input/output interface 95 and the communication interface 96 may be also used as an interface connected to an external device.

Input devices such as a keyboard, a mouse, and a touch panel may be connected to the information processing apparatus 90 as necessary. These input devices are used to input information and settings. In a case where the touch panel is used as the input device, a display screen of a display device may also serve as an interface of the input device. It is only necessary that the processor 91 and the input devices perform data communication via the input/output interface 95.

Furthermore, the information processing apparatus 90 may be provided with a display device for displaying information. In a case where a display device is provided, the information processing apparatus 90 preferably includes a display control device (not illustrated) for controlling display of the display device. It is only necessary to connect the display device to the information processing apparatus 90 via the input/output interface 95.

Furthermore, the information processing apparatus 90 may be provided with a drive device. The drive device mediates reading of data and a program from a recording medium, writing of a processing result of the information processing apparatus 90 in the recording medium, and the like, between the processor 91 and the recording medium (program recording medium). It is only necessary to connect the drive device to the information processing apparatus 90 via the input/output interface 95.

The above is an example of a hardware configuration for enabling control and processing according to each example embodiment of the present invention. The hardware configuration of FIG. 30 is an example of a hardware configuration for executing control and processing according to each example embodiment and does not limit the scope of the present invention. In addition, a program for causing a computer to execute control and processing according to each example embodiment is also included in the scope of the present invention. Further, a program recording medium in which the program according to each example embodiment is recorded is also included in the scope of the present invention. The recording medium can be implemented by, for example, an optical recording medium such as a compact disc (CD) or a digital versatile disc (DVD). The recording medium may be implemented by a semiconductor recording medium such as a universal serial bus (USB) memory or a secure digital (SD) card. Furthermore, the recording medium may be implemented by a magnetic recording medium such as a flexible disk, or other recording media. In a case where a program to be executed by the processor is recorded in a recording medium, the recording medium is relevant to the program recording medium.

The components of each example embodiment may be arbitrarily combined. In addition, the components of each example embodiment may be implemented by software or may be implemented by a circuit.

While the invention has been particularly shown and described with reference to exemplary example embodiments thereof, the invention is not limited to the above example embodiments. It will be understood by those of ordinary skill in the art that various changes in form and details may be made therein without departing from the spirit and scope of the present invention as defined by the claims.

Some or all of the above example embodiments may be described as the following supplementary notes, but are not limited to the following.

### (Supplementary Note 1)

A dynamic balance estimation device including:
a data acquisition unit that acquires feature amount data including a feature amount to be used for estimating dynamic balance of a user, the feature amount data being extracted from sensor data regarding motion of a foot of the user;
a storage unit that stores an estimation model that outputs a dynamic balance index according to an input of the feature amount data;
an estimation unit that inputs the acquired feature amount data to the estimation model to estimate the dynamic balance of the user in accordance with the dynamic balance index output from the estimation model; and
an output unit that outputs information on the estimated dynamic balance of the user.

### (Supplementary Note 2)

The dynamic balance estimation device according to supplementary note 1, in which the data acquisition unit acquires the feature amount data including a feature amount to be used for estimating a value of a result of a functional reach test as the dynamic balance index, the feature amount data being extracted from gait waveform data generated using time-series data of the sensor data regarding the motion of the foot.

### (Supplementary Note 3)

The dynamic balance estimation device according to supplementary note 2, in which
the storage unit stores, regarding the plurality of subjects, the estimation model generated by machine learning using teacher data having the feature amount to be used for estimating the dynamic balance index as an explanatory variable and having the dynamic balance indexes of the plurality of subjects as objective variables, and
the estimation unit inputs the feature amount data acquired regarding the user to the estimation model to estimate the dynamic balance of the user in accordance with the dynamic balance index of the user output from the estimation model.

### (Supplementary Note 4)

The dynamic balance estimation device according to supplementary note 3, in which the storage unit stores the estimation model machine-learned using explanatory variables including heights of the plurality of subjects, and
the estimation unit inputs the feature amount data and a height related to the user to the estimation model to estimate the dynamic balance of the user in accordance with the dynamic balance index of the user output from the estimation model.

### (Supplementary Note 5)

The dynamic balance estimation device according to supplementary note 3 or 4, in which the storage unit stores, regarding the gait waveform data of the plurality of subjects, the estimation model generated by machine learning using teacher data having a feature amount regarding an activity of a gluteus medius extracted from a load response period, a feature amount regarding an activity of an iliacus extracted from an initial swing period, a feature amount regarding a tibialis anterior and a short head of a biceps femoris extracted from a mid-swing period, and a feature amount regarding compensatory motion of a foot angle in a swing phase as explanatory variables, and having the dynamic balance indexes of the plurality of subjects as objective variables, and
the estimation unit inputs the feature amount data acquired in accordance with gait of the user to the estimation model to estimate the dynamic balance of the user in accordance with the dynamic balance index of the user output from the estimation model.

### (Supplementary Note 6)

The dynamic balance estimation device according to supplementary note 5, in which the storage unit stores, regarding the plurality of subjects, the estimation model generated by machine learning using teacher data having a feature amount extracted from a load response period of the gait waveform data of angular velocity in a coronal plane, a feature amount extracted from an initial swing period of the gait waveform data of acceleration in a vertical direction, a feature amount extracted from a mid-swing period of the gait waveform data of acceleration in a traveling direction, a feature amount extracted from a pre-swing period of the gait waveform data of an angle in a horizontal plane, and a feature amount related to a foot angle in a swing phase as explanatory variables, and having the dynamic balance indexes of the plurality of subjects as objective variables,
the data acquisition unit acquires the feature amount data including a feature amount in the load response period of the gait waveform data of the angular velocity in the coronal plane, a feature amount in the initial swing period of the gait waveform data of the acceleration in the vertical direction, a feature amount in the mid-swing period of the gait waveform data of the acceleration in the traveling direction, a feature amount in the pre-swing period of the gait waveform data of the angle in the horizontal plane, and a feature amount of the foot angle in the swing phase extracted in accordance with the gait of the user, and
the estimation unit inputs the acquired feature amount data to the estimation model to estimate the dynamic balance of the user in accordance with the dynamic balance index of the user output from the estimation model.

### (Supplementary Note 7)

The dynamic balance estimation device according to any one of supplementary notes 3 to 6, in which the estimation unit estimates information on the dynamic balance of the user in accordance with the dynamic balance index estimated regarding the user, and
the output unit outputs the estimated information on the dynamic balance.

### (Supplementary Note 8)

A dynamic balance estimation system including:
the dynamic balance estimation device according to any one of supplementary notes 1 to 7; and
a sensor that is installed on footwear of a user who is an estimation target of dynamic balance, measures spatial acceleration and spatial angular velocity, generates sensor data regarding motion of a foot using the measured spatial acceleration and spatial angular velocity, and outputs the generated sensor data, and a gait measurement device including a feature amount data generation unit that acquires time-series data of the sensor data including a feature of gait, extracts gait waveform data for one gait cycle from the time-series data of the sensor data, normalizes the extracted gait waveform data, extracts, from the normalized gait waveform data, a feature amount to be used for estimating the dynamic balance from a gait phase cluster constituted with at least one temporally consecutive gait phase, generates feature amount data including the extracted feature amount, and outputs the generated feature amount data to the dynamic balance estimation device.

### (Supplementary Note 9)

The dynamic balance estimation system according to supplementary note 8, in which the dynamic balance estimation device is implemented in a terminal device having a screen visible by the user, and
information on the dynamic balance estimated in accordance with the motion of the foot of the user is displayed on the screen of the terminal device.

### (Supplementary Note 10)

The dynamic balance estimation system according to supplementary note 9, in which the dynamic balance estimation device causes recommendation information according to the dynamic balance estimated in accordance with the motion of the foot of the user to be displayed on the screen of the terminal device.

### (Supplementary Note 11)

The dynamic balance estimation system according to supplementary note 10, in which the dynamic balance estimation device causes a video regarding training for training a body part related to the dynamic balance to be displayed on the screen of the terminal device as the recommendation information according to the dynamic balance estimated in accordance with the motion of the foot of the user.

### (Supplementary Note 12)

A dynamic balance estimation method to be performed by a computer, the dynamic balance estimation method including:
acquiring feature amount data including a feature amount to be used for estimating dynamic balance of a user, the feature amount data being extracted from sensor data regarding motion of a foot of the user;
inputting the acquired feature amount data to an estimation model that outputs a dynamic balance index according to an input of the feature amount data;
estimating the dynamic balance of the user in accordance with the dynamic balance index output from the estimation model; and
outputting information on the estimated dynamic balance of the user.

### (Supplementary Note 13)

A program that causes a computer to execute:
processing of acquiring feature amount data including a feature amount to be used for estimating dynamic balance of a user, the feature amount data being extracted from sensor data regarding motion of a foot of the user;
processing of inputting the acquired feature amount data to an estimation model that outputs a dynamic balance index according to an input of the feature amount data;
processing of estimating the dynamic balance of the user in accordance with the dynamic balance index output from the estimation model; and
processing of outputting information on the estimated dynamic balance of the user.

### REFERENCE SIGNS LIST

1 dynamic balance estimation system
2 machine learning system
10, 20 gait measurement device
11 sensor
12 feature amount data generation unit
13 dynamic balance estimation device
25 machine learning device
111 acceleration sensor
112 angular velocity sensor
121 acquisition unit
122 normalization unit
123 extraction unit
125 generation unit
127 feature amount data output unit
131, 331 data acquisition unit
132, 332 storage unit
133, 333 estimation unit
135, 335 output unit
251 reception unit
253 machine learning unit
255 storage unit

What is claimed is:

1. A dynamic balance estimation device comprising:
a storage configured to store an estimation model that outputs a dynamic balance index according to an input of feature amount data used for estimating dynamic balance;
a memory storing instructions; and
a processor connected to the memory and configured to execute the instructions to:
acquire feature amount data including a feature amount used for estimating dynamic balance of a user, the feature amount data being extracted from sensor data regarding motion of a foot of the user;
input the acquired feature amount data to the estimation model to estimate the dynamic balance of the user in accordance with the dynamic balance index output from the estimation model; and
output information on the estimated dynamic balance of the user.

2. The dynamic balance estimation device according to claim 1, wherein
the processor is configured to execute the instructions to
acquire the feature amount data including a feature amount to be used for estimating a value of a result of a functional reach test as the dynamic balance index, the feature amount data being extracted from gait waveform data generated using time-series data of the sensor data regarding the motion of the foot.

3. The dynamic balance estimation device according to claim 2, wherein
the storage stores, regarding a plurality of subjects, the estimation model generated by machine learning using teacher data having the feature amount to be used for estimating the dynamic balance index as an explanatory variable and having the dynamic balance indexes of the plurality of subjects as objective variables, and
the processor is configured to execute the instructions to input the feature amount data acquired regarding the user to the estimation model to estimate the dynamic balance of the user in accordance with the dynamic balance index of the user output from the estimation model.

4. The dynamic balance estimation device according to claim 3, wherein
the storage stores the estimation model machine-learned using explanatory variables including heights of the plurality of subjects, and
the processor is configured to execute the instructions to input the feature amount data and a height related to the user to the estimation model to estimate the dynamic balance of the user in accordance with the dynamic balance index of the user output from the estimation model.

5. The dynamic balance estimation device according to claim 3, wherein
the storage stores, regarding the gait waveform data of the plurality of subjects, the estimation model generated by machine learning using teacher data having a feature amount regarding an activity of a gluteus medius extracted from a load response period, a feature amount regarding an activity of an iliacus extracted from an initial swing period, a feature amount regarding a tibialis anterior and a short head of a biceps femoris extracted from a mid-swing period, and a feature amount regarding compensatory motion of a foot angle in a swing phase as explanatory variables, and having the dynamic balance indexes of the plurality of subjects as objective variables, and
the processor is configured to execute the instructions to input the feature amount data acquired in accordance with gait of the user to the estimation model to estimate the dynamic balance of the user in accordance with the dynamic balance index of the user output from the estimation model.

6. The dynamic balance estimation device according to claim 5, wherein
the storage stores, regarding the plurality of subjects, the estimation model generated by machine learning using teacher data having a feature amount extracted from a load response period of the gait waveform data of angular velocity in a coronal plane, a feature amount extracted from an initial swing period of the gait waveform data of acceleration in a vertical direction, a feature amount extracted from a mid-swing period of the gait waveform data of acceleration in a traveling direction, a feature amount extracted from a pre-swing period of the gait waveform data of an angle in a horizontal plane, and a feature amount related to a foot angle in a swing phase as explanatory variables, and having the dynamic balance indexes of the plurality of subjects as objective variables,
the processor is configured to execute the instructions to
acquire the feature amount data including a feature amount in the load response period of the gait waveform data of the angular velocity in the coronal plane, a feature amount in the initial swing period of the gait waveform data of the acceleration in the vertical direction, a feature amount in the mid-swing period of the gait waveform data of the acceleration in the traveling direction, a feature amount in the pre-swing period of the gait waveform data of the angle in the horizontal plane, and a feature amount of the foot angle in the swing phase extracted in accordance with the gait of the user, and
input the acquired feature amount data to the estimation model to estimate the dynamic balance of the user in accordance with the dynamic balance index of the user output from the estimation model.

7. The dynamic balance estimation device according to claim 3, wherein
the processor is configured to execute the instructions to
estimate information on the dynamic balance of the user in accordance with the dynamic balance index estimated regarding the user, and
output the estimated information on the dynamic balance.

8. A dynamic balance estimation system comprising:
the dynamic balance estimation device according to claim 1; and
a gait measurement device including:
a sensor that is installed on footwear of a user who is an estimation target of dynamic balance, measures spatial acceleration and spatial angular velocity, generates sensor data regarding motion of a foot using the measured spatial acceleration and spatial angular velocity, and outputs the generated sensor data; and
a memory storing instructions; and
a processor connected to the memory and configured to execute the instructions to:
acquire time-series data of the sensor data including a feature of gait,
extract gait waveform data for one gait cycle from the time-series data of the sensor data,
normalize the extracted gait waveform data,
extract, from the normalized gait waveform data, a feature amount to be used for estimating the dynamic balance from a gait phase cluster constituted with at least one temporally consecutive gait phase,
generate feature amount data including the extracted feature amount, and
output the generated feature amount data to the dynamic balance estimation device.

9. The dynamic balance estimation system according to claim 8, wherein
the dynamic balance estimation device is implemented in a terminal device having a screen visible by the user, and
information on the dynamic balance estimated in accordance with the motion of the foot of the user is displayed on the screen of the terminal device.

10. The dynamic balance estimation system according to claim 9, wherein
the processer of the dynamic balance estimation device is configured to execute the instructions to cause recommendation information according to the dynamic balance estimated in accordance with the motion of the foot of the user to be displayed on the screen of the terminal device.

11. The dynamic balance estimation system according to claim 10, wherein
the processer of the dynamic balance estimation device is configured to execute the instructions to cause a video regarding training for training a body part related to the dynamic balance to be displayed on the screen of the terminal device as the recommendation information according to the dynamic balance estimated in accordance with the motion of the foot of the user.

12. A dynamic balance estimation method to be performed by a computer, the dynamic balance estimation method comprising:
acquiring feature amount data including a feature amount to be used for estimating dynamic balance of a user, the feature amount data being extracted from sensor data regarding motion of a foot of the user;
inputting the acquired feature amount data to an estimation model that outputs a dynamic balance index according to an input of the feature amount data;
estimating the dynamic balance of the user in accordance with the dynamic balance index output from the estimation model; and
outputting information on the estimated dynamic balance of the user.

13. A non-transitory recording medium recording a program that causes a computer to execute:
processing of acquiring feature amount data including a feature amount to be used for estimating dynamic balance of a user, the feature amount data being extracted from sensor data regarding motion of a foot of the user;
processing of inputting the acquired feature amount data to an estimation model that outputs a dynamic balance index according to an input of the feature amount data;
processing of estimating the dynamic balance of the user in accordance with the dynamic balance index output from the estimation model; and
processing of outputting information on the estimated dynamic balance of the user.

14. The dynamic balance estimation system according to claim 10, wherein
the processor of the dynamic balance estimation device is configured to execute the instructions to
cause the recommendation information that supports the user for making decision about taking an action to be displayed on the screen of the terminal device.
